(19) United States
(12) Patent Application Publication
(10) Pub. No.: US 2021/0008110 A1
(43) Pub. Date: Jan. 14, 2021

(54) ACTIVATED LYMPHOCYTIC CELLS AND METHODS OF USING THE SAME TO TREAT CANCER AND INFECTIOUS CONDITIONS

(71) Applicant: Serhat Gumrukcu, (US)

(72) Inventor: Serhat Gumrukcu, Los Angeles, CA (US)

(21) Appl. No.: 16/901,427

(22) Filed: Jun. 15, 2020

Related U.S. Application Data
(60) Provisional application No. 62/861,487, filed on Jun. 14, 2019.

(51) Int. Cl. A61K 35/17; A61K 31/7076; A61K 31/664; A61K 38/21; A61K 38/20; A61K 31/5415; A61K 31/138; A61P 35/00; A61P 31/18; A61K 31/415; A61K 31/616

(52) U.S. Cl. CPC A61K 35/17; A61K 31/7076; A61K 31/664; A61K 38/212; A61K 38/2013; A61K 31/616; A61K 31/138; A61P 35/00; A61P 31/18; A61K 31/415; A61K 31/5415

(57) ABSTRACT

Provided herein are methods for treating a patient with HIV, cancer, a viral infection, or a bacterial infection, comprising administering an effective amount of activated lymphocytic cellular compositions. Related compositions, kits, and methods for modulating the immune system using the activated lymphocytic cellular compositions are also provided.

### CROSS REFERENCE TO RELATED APPLICATIONS

This application claims priority to U.S. Provisional Application No. 62/861,487, filed Jun. 14, 2019, which is hereby incorporated by reference in its entirety.

### FIELD

Provided herein are methods for treating a patient with human immunodeficiency virus (HIV), cancer, a viral infection, or a bacterial infection, comprising administering cellular compositions comprising activated lymphocytic cells, such as natural kill (NK) cells. Related compositions, kits, and methods for modulating the immune system using such activated cells are also provided.

### BACKGROUND

Natural killer (NK) cells are innate lymphocytes important for mediating anti-viral and anti-cancer immunity through cytokine and chemokine secretion, and through the release of cytotoxic granules (Vivier et al. Science 331(6013):44-49 (2011); Caligiuri, Blood 112(3):461-469 (2008); Roda et al., Cancer Res. 66(1):517-526 (2006)). Thus, NK cells are part of the body's defense system and act as an army to protect from invaders like viruses and infections. Similarly, NK cells are important immune effector cells for fighting cancer.

Early clinical trials using autologous NK cells failed to demonstrate significant clinical benefit. Since NK cells in cancer patients are highly dysfunctional and reduced in number, methods and compositions for restoring or increasing cytolytic NK cell numbers and increasing their anti-tumor, anti-viral, and/or anti-bacterial activity are greatly needed.

### SUMMARY

In some embodiments, methods of treating a patient with HIV, cancer, a viral infection, or a bacterial infection, the method comprising administering an effective amount of a lymphocytic cellular composition comprising activated NK cells to the patient are provided.

In some embodiments, methods for increasing immune response in a patient in need thereof, comprising administering an effective amount of a lymphocytic cellular composition comprising activated NK cells to the patient are provided.

In some embodiments, methods of inhibiting HIV replication in a patient, the method comprising administering to the subject with HIV an effective amount of a lymphocytic cellular composition comprising activated natural killer (NK) cells to the patient are provided.

In some embodiments, methods of inhibiting tumor growth in a patient, the method comprising administering to the patient with the tumor an effective amount of a lymphocytic cellular composition comprising activated natural killer (NK) cells to the patient are provided.

In some embodiments, methods of inhibiting a viral or bacterial replication or reproduction in a subject having a viral or bacterial infection, the method comprising administering to the subject with the viral or bacterial infection an effective amount of a lymphocytic cellular composition comprising activated natural killer (NK) cells to the patient are provided.

In some embodiments, methods of activating NK cells, the method comprising contacting a cellular composition comprising the NK cells in vitro with at least one cytokine and optionally soluble fibroblast growth factor receptor 1(sFGFR1) are provided.

In some embodiments, a cytokine and optionally soluble fibroblast growth factor receptor 1 (sFGFR1) activated lymphocytic cellular composition comprising activated natural killer (NK) cells is provided. In some embodiments, the composition is a pharmaceutical composition.

In some embodiments, a cytokine and optionally soluble fibroblast growth factor receptor 1 (sFGFR1) activated lymphocytic cellular composition comprising activated natural killer (NK) cells for treating a patient with HIV, cancer, a viral infection, or a bacterial infection is provided. In some embodiments, the composition is a pharmaceutical composition.

In some embodiments, a cytokine and optionally soluble fibroblast growth factor receptor 1 (sFGFR1) activated lymphocytic cellular composition comprising activated natural killer (NK) cells for the preparation of a medicament for treating a patient with HIV, cancer, a viral infection, or a bacterial infection are provided. In some embodiments, the composition is a pharmaceutical composition.

In certain embodiments, the cellular compositions further comprise activated Gamma delta T cells (GDT cells). In some embodiments, the cellular compositions further comprise invariant natural killer T cells (iNKT cells). In some embodiments, the cellular compositions further comprise CD3 T cells.

In some embodiments, prior to administration to the patient, the lymphocytic cellular composition is activated by mixing/incubating/contacting the cells with at least one cytokine and optionally soluble fibroblast growth factor receptor 1 (sFGFR1).

In some embodiments, the cytokine is selected from the group consisting of IL-2, IL-15, IL-21, Flt3-L, stem cell factor (SCF), IL-7, IL-12, and IL18, and any combination thereof.

In some embodiments, the cytokine is incubated/mixed/contacted with the cellular composition for between about 6-24 hours.

In some embodiments, the amount of cytokine incubated with the cellular composition is between about 100-1000 IU/ml.

In some embodiments, the method further comprises pre-conditioning the patient prior to administering the activated NK cells by administering at least one lympho-suppressive agent, chemotherapeutic agent, or immunosuppressive agent for between 3-5 days prior to administering the lymphocytic cellular composition.

In yet additional embodiments, the lympho-suppressive or chemotherapeutic agent comprises any one or combination of agents including 6TG, 6-MMP, and one or more purine analogues selected from the group consisting of clofarabine, fludarabine, and cytarabine.

In some embodiments, the dose of the lympho-suppressive or chemotherapeutic agent ranges from about 5 mg/m² to about 50 mg/m².

In some embodiments, the chemotherapeutic or immunosuppressive agent comprises any one or a combination of cyclophosphamide, rituximab, and optionally a steroid.

In some embodiments, the steroid is a glucocorticoid steroid.

In some embodiments, the steroid is prednisone.

In some embodiments, the dose of the chemotherapeutic agent or immunosuppressive agent ranges from about 20 mg/m² to about 1000 mg/m² (confirm lowest and highest possible dosages).

In some embodiments, the method further comprises administering to the patient interferon-alpha (IFN-α), or a biological equivalent thereof, at a dosage of between about 1×10⁶ IU/m²/d to about 10×10⁶ IU/m²/d, two days and 1 day prior to administering the lymphocytic cellular composition comprising activated NK cells.

In some embodiments, the method further comprises administering to the patient one or two antihistamine drugs, on the same day, but at least about 2-6 hours prior to administration of the lymphocytic cellular composition comprising activated NK cells to the patient.

In some embodiments, the method further comprises administering to the patient IL-2 on the day of administration and on the first day following administration of the lymphocytic cellular composition comprising activated NK cells, and continuing for between 3-14 additional days following administration of the activated NK cells, in a dosage of about 3-6×10⁶ IU per dose.

In some embodiments, the method further comprises administering to the patient a COX-2 inhibitor or a nonsteroidal anti-inflammatory drug (NSAID), or a combination thereof, on the day of administration of the activated NK cells, and continuing for between at least 14 and 60 days following administration of the activated NK cells.

In some embodiments, the COX-2 inhibitor is celecoxib or rofecoxib, and the nonsteroidal anti-inflammatory drug is selected from the group consisting of aspirin, indomethacin (Indocin), ibuprofen (Advil, Motrin), naproxen (Naprosyn), piroxicam (Feldene), and nabumetone (Relafen).

In some embodiments, the NK cells are allogeneic to the patient.

In some embodiments, the NK cells are not HLA-matched with the patient. In some embodiments, the NK cells are intentionally HLA-mismatched, or MR-inhibitory ligand/HLA mismatched.

In some embodiments, the dosage of activated cells ranges from about 3-50×10⁶.

In some embodiments, the viral infection is selected from the group consisting of viral infections caused by retroviruses (e.g., human T-cell lymphotrophic virus (HTLV) types I and II and human immunodeficiency virus (HIV)), herpes viruses (e.g., herpes simplex virus (HSV) types I and II, Epstein-Ban virus and cytomegalovirus), arenaviruses (e.g., lassa fever virus), paramyxoviruses (e.g., morbillivirus virus, human respiratory syncytial virus, and pneumovirus), adenoviruses, bunyaviruses (e.g., hantavirus), coronaviruses, filoviruses (e.g., Ebola virus), flaviviruses (e.g., hepatitis C virus (HCV), yellow fever virus, and Japanese encephalitis virus), hepadnaviruses (e.g., hepatitis B viruses (HBV)), orthomyoviruses (e.g., Sendai virus and influenza viruses A, B and C), papovaviruses (e.g., papillomaviruses), picornaviruses (e.g., rhinoviruses, enteroviruses and hepatitis A viruses), poxviruses, reoviruses (e.g., rotaviruses), togaviruses (e.g., rubella virus), and rhabdoviruses (e.g., rabies virus), and any combination thereof. In some embodiments, the viral infection is HIV. In some embodiments, the viral infection is HCV. In some embodiments, the viral infection is HBV. In some embodiments, the viral infection is a coronavirus. In some embodiments, the viral infection is COVID-19.

In some embodiments, the cancer is a hematological or hematogenous cancer selected from the group consisting of acute leukemia, acute myelocytic leukemia, acute myelogenous leukemia, myeloblastic leukemia, promyelocytic leukemia, myelomonocytic leukemia, monocytic leukemia, erythro leukemia, chronic leukemia, chronic myelocytic (or granulocytic) leukemia, chronic myelogenous leukemia, chronic lymphocytic leukemia, polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia, and any combination thereof.

In some embodiments, the cancer is a solid tumor selected from the group consisting of fibrosarcoma, myxosarcoma, liposarcoma, chondrosarcoma, osteosarcoma, and other sarcomas, synovioma, mesothelioma, Ewing's tumor, leiomyosarcoma, rhabdomyo sarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases.

In some embodiments, the bacterial infection is caused by one of the pathogenic bacterial species Bacteroides, Clostridium, Streptococcus, Staphylococcus, Pseudomonas, Haemophilus, Legionella, Mycobacterium, Escherichia, Salmonella, Shigella, or Listeria.

In some embodiments, graft versus host disease (GVHD) is decreased or eliminated, while graft versus tumor (GVT) or graft versus virus (GVV) is increased in the patient.

In some embodiments, administration of IL-2 is counter-indicated and not administered following administration of the lymphocytic cellular composition comprising activated NK cells, when the patient has lymphocytic or lymphoblastic leukemia or lymphoma.

In some embodiments, the method further comprises at a time-frame of from 4-14 days following administration of the lymphocytic cellular composition comprising activated NK cells, administering the patient an immune check-point inhibitor, including any one or combination of two check point inhibitors, including an inhibitor of PD-1 or PD-L1 (B7-H1), such as an anti-PD-1 antibody, including nivolumab (Nivolumab from Bristol-Myers Squibb), pembrolizumab/lambrolizumab, also known as MK-3475 (Keytruda from Merck), pidilizumab (Curetech), AMP-224 (Amplimmune), or an anti-PD-L1 antibody, including MPDL3280A (Roche), MDX-1105 (Bristol Myer Squibb), MEDI-4736 (Astra7eneca) and MSB-0010718 C (Merck), an antagonist of CTLA-4, such as an anti-CTLA-4 antibody including anti-CTLA4 antibody Yervoy™ (ipilimumab, Bristol-Myers Squibb), tremelimumab (Pfizer), Ticilimumab (AstraZeneca) or AMGP-224 (Glaxo Smith Kline), or a tumor specific antibody trastuzumab (Herceptin) for breast cancer, rituximab (Rituxan) for lymphoma, or cetuximab (Erbitux).

In some embodiments, the treatment or increasing the immune response is repeated periodically for time frames of from once every month, to once every two months, to once every 3 months, to once every 4 months, to once every 5 months, to once every 6 months, or once every 7 months, or once every 8 months, or once every 9 months, or once every 10 months, or once every 11 months, or once annually as a maintenance treatment, for as long as the patient exhibits improvement or stable/non-progressing disease.

### BRIEF DESCRIPTION OF THE DRAWINGS

FIG. 1 is a table showing exemplary treatment regimens including pre-treatment conditioning, cell infusion treatments, and post-cell infusion treatment options (“post-conditioning”) for the various patient populations.

FIG. 2A illustrates a non-limiting treatment regimen and HIV viral load counts from an HIV patient treated according to the methods described herein.

FIG. 2B illustrates a non-limiting treatment regimen and HIV viral load counts from an HIV patient treated according to the methods described herein.

FIG. 3A, FIG. 3B, FIG. 3C, FIG. 3D, and FIG. 3E illustrate tables and schematics summarizing patient numbers and tumor types (FIG. 3A), treatment regimens/protocols (FIG. 3B), NK cell characterizations (FIG. 3C) treatment toxicities observed (FIG. 3D) and survival data (FIG. 3E).

FIG. 4 illustrates a plasmid map showing the sequence of the extracellular component of sFGFR1 and expression vector.

FIG. 5 is a diagram showing cancer-immunity cycle.

FIG. 6A, FIG. 6B, FIG. 6C, and FIG. 6D are flow cytometry images of NK cell preparations at the end of 72 hours of activation using no activator (FIG. 6A), IL-2 as the activator (FIG. 6B), FGFR1 as the activator (FIG. 6C), and the combination of IL-2 and FGFR1 as activators (FIG. 6D).

### DETAILED DESCRIPTION

NK cells are innate immune cells that form the first line of defense against viruses and tumors. While significant advances have been made in cancer treatment by use of antibodies directed against cancer antigens, the responsiveness of patients to such antibodies varies. Investigation of such variable responses has typically focused on the direct inhibitory effects of these antibodies on the tumor cells (e.g. inhibition of growth factor receptors and the subsequent induction of apoptosis) while the in vivo effects of these antibodies may be more complex and may involve the host immune system. For example, the mechanism of action of such anti-cancer antibodies may include one or more of the following: antibody-dependent cellular cytotoxicity (ADCC), antibody-dependent cytokine/chemokine production, and complement-dependent cytotoxicity (CMC). (See, review Veluchamy, John, et al. Front Immunol. 2017; 8:631).

Antibody-dependent cellular cytotoxicity and antibody-dependent cytokine/chemokine production are primarily mediated by the specialized subset of lymphocytes, natural killer (NK) cells. NK cells are effector cells that comprise the third largest population of lymphocytes and are important for host immuno-surveillance against tumor and pathogen-infected cells. NK Cells and their Activating and Inhibitory Receptors

Human NK cells are generally categorized by their level of CD56 and CD16 expression into two subsets: CD56^{bright}CD16^{dim} and CD56^{dim}CD16^{bright}NK cells. Most NK cells in the peripheral blood and spleen are CD56^{dim}CD16^{bright} and are cytotoxic against a variety of tumor cells, whereas CD56^{bright}CD16^{dim}NK cells are immune regulatory in function and constitute the majority in secondary lymphoid tissues, producing abundant cytokines but exerting weak cytotoxicity compared to CD56^{dim}CD16^{bright} cells (See, Cooper, M. A., et al. Trends Immunol (2001) 22(11):633-40). The ability of NK cells to discriminate between a cancer cell and a healthy cell is regulated, at least in part, by a balance between its activating and inhibitory receptors. NK-activating receptors such as DNAM-1 and NKG2D; natural cytotoxicity receptors (NCRs) such as NKp30, NKp44, NKp46, CD94/NKG2C, CD94/NKG2E, and CD16a; and activating killer cell-immunoglobulin like receptors (KIRs) contribute to NK cell activation, triggering the release of cytotoxic granules and proinflammatory cytokines such as interferon gamma (IFNγ) from NK cells to lyse cancer cells. The NK cell-activating receptor NKG2D (CD314) recognizes MHC class-I-chain related proteins A and B (MICA and MICB) and ULBPs, while DNAM-1 binds to CD112 (Nectin-2) and CD155 (poliovirus receptor) on stressed, infected, and cancer cells. The ligands for NCRs are widely expressed on cells infected by viruses or by intracellular bacteria and on tumor cells, but their exact modes of action are yet to be characterized to define their role in NK cytotoxicity. NKG2, also known as CD159 (Cluster of Differentiation 159) is a receptor for natural killer cells, There are 7 NKG2 types: A, B, C, D, E, F and H. NKG2D is an activating receptor on the NK cell surface. NKG2A dimerizes with CD94 to make an inhibitory receptor (CD94/NKG2). Heterodimers of the NKG2 family; CD94/NKG2C and CD94/NKG2E recognize the non-classical MHC class I molecule HLA-E and associate with DAP-12 molecule to trigger an NK activation signal (See, Borrego, F. et al, J Exp Med (1998) 187(5):813-8; and Pegram, H. J. et al. Immunol Cell Biol (2011) 89(2):216-24). Another important activation mechanism of NK cells is through the interaction of CD16a (FcγRIIIa, a low affinity Fc receptor) with the Fc portion of IgG1 antibodies, forming an immunological synapse to engage antibody opsonized targets for NK cell-mediated antibody-dependent cell mediated cytotoxicity (ADCC) (Leibson, P. J. Immunity (1997) 6(6):655-61). Besides engaging activating receptors, NK cells also induce target cell death using tumor necrosis factor α (TNF-α), Fas ligand, and TNF-related apoptosis-inducing ligand (TRAIL). The most prominent NK cell inhibitory receptors include inhibitory KIRs that recognize MHC class I (HLA-ABC) molecules, which are universally expressed on healthy tissues. Similarly, CD94/NKG2A, an inhibitory receptor from the NKG2 family, binds to HLA-E and induces NK cell tolerance through the activation of an intracellular immunoreceptor tyrosine-based inhibitory motif (ITIM). Hence, knowing that NK cell functions are determined by an array of receptors, which can either potentiate an activating or inhibitory signal, depending on different ligand interactions with tumor cells, it is important to shift the balance in a therapeutic setting toward an activating NK phenotype to expedite enhanced NK tumor killing mechanisms.

### NK Cell Dysfunctionality in Cancer

Natural killer cells can control circulating tumor cells and prevent formation of tumor metastases. However, tumors employ different strategies to evade killing by NK cells. Upregulation of inhibitory ligands such as MHC class I molecules (HLA-ABC, HLA-G and HLA-E) has been associated with a stronger inhibitory signal to NK cells. Furthermore, increased expression of the inhibitory NKG2A receptor reported in renal cell carcinoma resulted in decreased functionality of tumor infiltrating NK cells. On the other hand, downregulation of NK-activating ligands for NKG2D such as MICA and MICB and increased shedding of tumor-derived soluble MIC also impair NKG2D-mediated NK cell tumor recognition. Another important feature for optimal NK cell function is the ability to home and migrate to tumor sites. Several studies have correlated increased homing of NK cells to tumor tissues with improved treatment outcomes in solid tumors. However, the immunosuppressive tumor stroma comprising regulatory T cells (T-regs), myeloid-derived suppressor cells (MDSCs), M2 macrophages, and immature dendritic cells severely restricts NK cell functionality and their entry into solid tumors. In chronic diseases, such as those associated with human immunodeficiency virus and cytomegalovirus infections, mainly exhausted NK cells with decreased cytokine production and reduced cytolytic activity are observed. In a study with breast cancer patients, the NK cell expression levels of activating receptors (NKG2D, DNAM, CD16, and NKp30) were decreased, whereas inhibitory receptor (NKG2A) expression levels were increased and this apparent dysfunctionality of NK cells was found to directly affect NK cell cytotoxicity. Similarly, the effector subset of NK cells (CD56dimCD16+) from head and neck and breast cancer patients, when tested in vitro, was highly prone to apoptosis, thus pointing to low NK cell activity in these patients. Impaired NK cell functionality may result from tumor-imposed suppressive mechanisms and presents a major hurdle for NK cell-targeted immunotherapies. Therefore, approaches to restore or replace impaired NK cell cytotoxicity will serve as effective therapies for increasing the modulating host defenses against cancers, as well as bacterial and viral infections such as HIV.

Unlike autologous NK cells, allogeneic NK cells are not restricted by the patient's tumor's HLA expression, which is an added advantage to mount an improved anti-tumor effect.

Upon activation, NK cells produce cytokines and chemokines abundantly and at the same time exhibit potent cytolytic activity. An advantage of this NK activation is that it is major histocompatibility complex (MHC) independent. Activation of NK cells can occur through the direct binding of NK cell receptors to ligands on the target cell, as seen with direct tumor cell killing, or through the crosslinking of the Fc receptor (CD16; FcγRIII) by binding to the Fc portion of antibodies bound to an antigen-bearing cell. This CD16 engagement (CD16 crosslinking) initiates NK cell responses via intracellular signals that are generated through one, or both, of the CD16-associated adaptor chains, FcRγ or CD3ζ. Triggering of CD16 leads to phosphorylation of the gamma or zeta chain, which in turn recruits tyrosine kinases, syk and ZAP-70, initiating a cascade of signal transduction leading to rapid and potent effector functions. The most well-known effector function is the release of cytoplasmic granules carrying toxic proteins to kill nearby target cells through the process of antibody-dependent cellular cytotoxicity (ADCC). CD16 crosslinking also results in the production of cytokines and chemokines that, in turn, activate and orchestrate a series of immune responses.

This release of cytokines and chemokines is likely to play a role in the anti-cancer activity of activated NK cells in vivo. NK cells also have small granules in their cytoplasm containing perforin and proteases (granzymes). Upon release from the activated NK cell, perforin forms pores in the cell membrane of targeted cells through which the granzymes and associated molecules can enter, inducing apoptosis. The fact that activated NK cells induce apoptosis rather than necrosis of target cells is significant—necrosis of a virus-infected cell would release the virions, whereas apoptosis leads to destruction of the virus inside the cells.

The expression and signal transduction activity of several NK cell activation receptors requires physically associated adaptors, which transduce signals through immunoreceptor tyrosine-based activation motifs (ITAMs). Among these adaptors, FcRγ and CD3ζ chains can associate with CD16 and natural cytotoxicity receptors (NCRs) as either disulfide-linked homo-dimers or hetero-dimers, and these chains have been thought to be expressed by all mature NK cells.

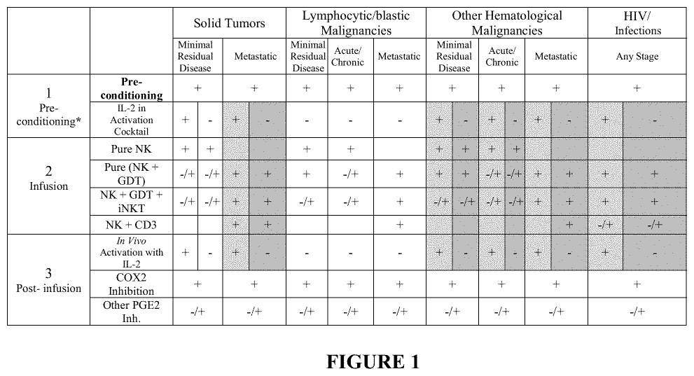

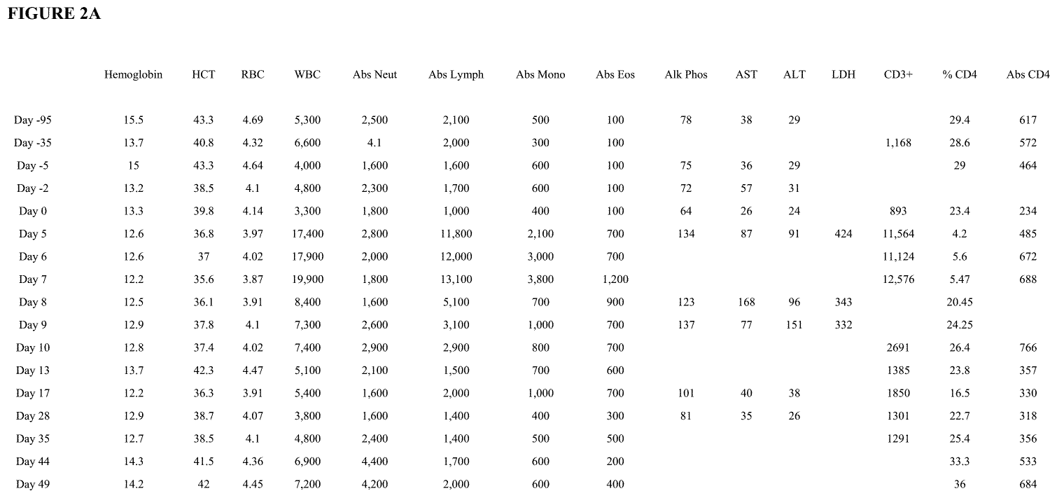

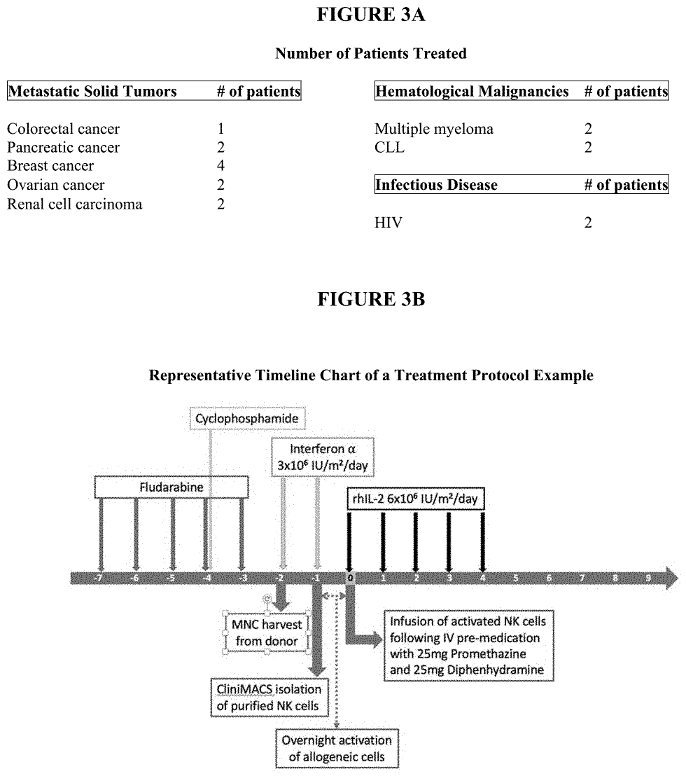

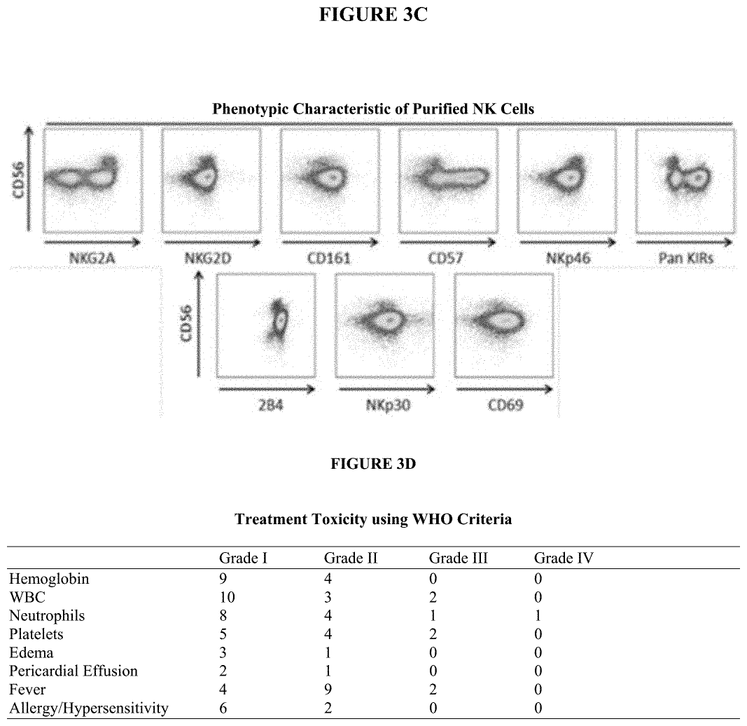

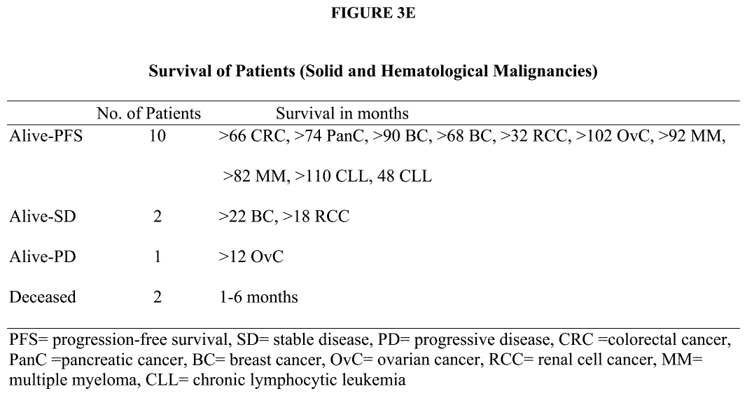

Thus, while not wishing to be bound by theory, aspects of the present invention relates to providing a lymphocytic cellular composition comprising an effective amount of activated NK cells, or a combination of activated NK cells in combination with an effective amount of any of the following: activated Gamma delta T cells (GDT cells); activated GDT cells and iNKT cells; or CD3 T cells, to a patient in a regimen that combines specific pre-conditioning and post-cell infusion conditioning steps (See FIG. 1), to provide the patient with an improved immune response to cancer or infectious disease, including to HIV. The methods and compositions described herein have many advantages over previous methods, including the lack of co-culturing on feeder cells, and the ability to alleviate unwanted T regulatory effects, as well as to minimize risks of contamination of the cellular composition, and ease and speed of preparing and delivering the activated cellular composition to the patient, along with very minimal side effects from the cellular composition, which are typically only Grade 1 or less side effects, See FIG. 3D). These methods also serve to provide a “vaccine effect” training host, patient immune cells to similarly recognize and kill the cancer, bacterial, or viral targets.

### Abbreviations:

Antibody-dependent cellular cytotoxicity: ADCC

Cluster differentiation 3: CD3

Graft-versus-tumor effects: GVT

Graft versus host disease: GVHD

Graft versus virus: GVV

Gamma delta T cells: GDT cells, also γδT cells.

Human immunodeficiency virus (HIV): A lentivirus that causes acquired immunodeficiency syndrome.

Invariant natural killer T cells: iNK T cells, also known as type I or classical NKT cells, are a distinct population of T cells that express an invariant aβ T-cell receptor (TCR) and a number of cell surface molecules in common with natural killer (NK) cells.

Natural Killer cells: NK cells

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein and unless otherwise indicated, the term “about” is intended to mean ±5% of the value it modifies. Thus, about 100 means 95 to 105. Additionally, the term “about” modifies a term in a series of terms, such as “about 1, 2, 3, 4, or 5” it should be understood that the term “about” modifies each of the members of the list, such that “about 1, 2, 3, 4, or 5” can be understood to mean “about 1, about 2, about 3, about 4, or about 5.” The same is true for a list that is modified by the term “at least” or other quantifying modifier, such as, but not limited to, “less than,” “greater than,” and the like.

As used herein and in the appended claims, the singular forms “a”, “an” and “the” include plural reference unless the context clearly dictates otherwise.

As used herein, the terms “comprise,” “have,” “has,” and “include” and their conjugates, as used herein, mean “including but not limited to.” While various compositions, and methods are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

The term “soluble FGFR1,” as used herein, refers to vertebrate fibroblast growth factor receptor 1 and in particular the protein from Homo sapiens, (UNIPROTKB:P11362-7, complete information can be found on the world wide web at: genecards.org/cgi-bin/carddisp.pl?gene=FGFR1). The vertebrate fibroblast growth factor receptor (FGFR) family is an important group of proteins involved in embryonic development and the growth and proliferation of adult cells. Mutations in FGFR proteins can lead to pathologies including bone or limb defects and various forms of cancer. FGFR proteins are receptor tyrosine kinases that, upon ligand binding, dimerize and signal through the MAPK and PLCγ pathways. FGFR1 is a well-characterized member of this protein family consisting of an extracellular region, a single-pass transmembrane domain, and the intracellular tyrosine kinase domain. The extracellular region contains a heparin binding domain responsible for interaction with the extracellular matrix while the intracellular domain interacts with downstream effectors after receptor dimerization to propagate signals (Int. J. Dev. Biol. 2002;46(4):393-400). FGFR1 exists in many alternatively spliced isoforms including a soluble, secreted isoform lacking the transmembrane and kinase domains. While their function is unclear, soluble FGFR1 isoforms may bind to and regulate the activity of FGF ligands during development (Int. J. Dev. Biol. 2002;46(4):393-400). Typical FGFR1 signaling occurs after ligand binding, receptor dimerization, and phosphorylation of downstream effector proteins. However, recent research has shown internalized FGFR1 is transported to the nucleus where it regulates target gene expression and cell proliferation in cancer cells (J. Cell Biol. 2012 Jun. 11;197(6):801-17). (See, also for example: Breast Cancer Res. 2012 Aug 3;14(4):R115).

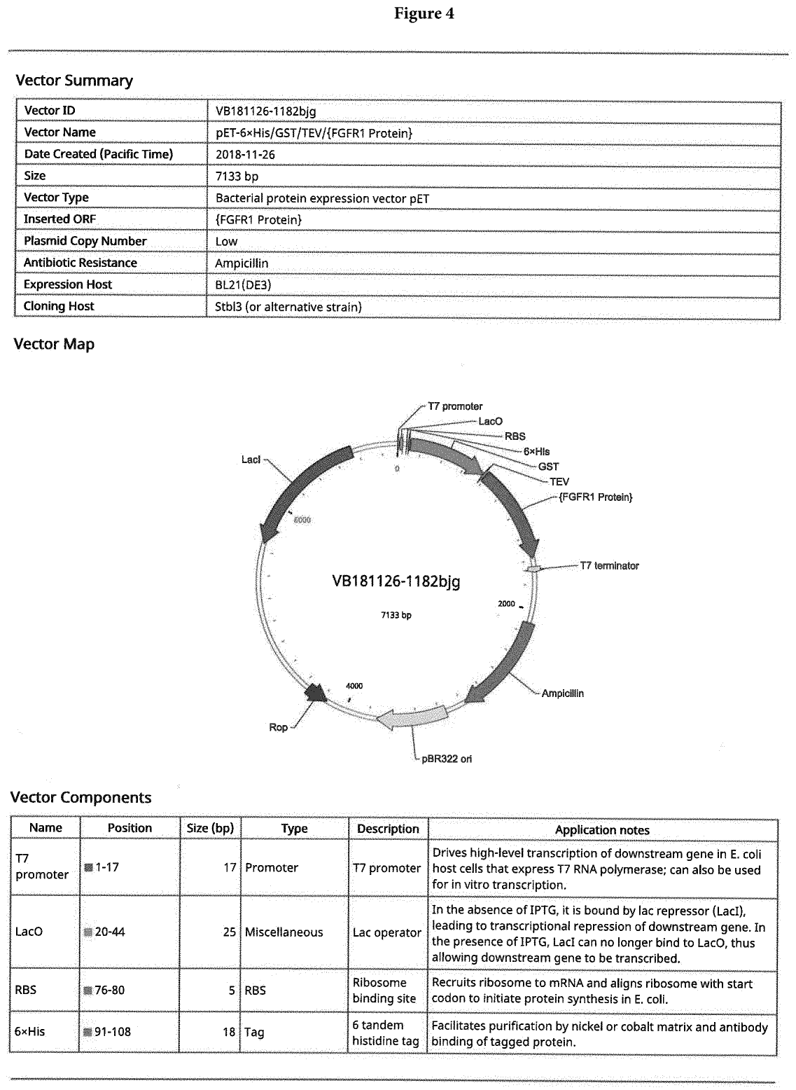

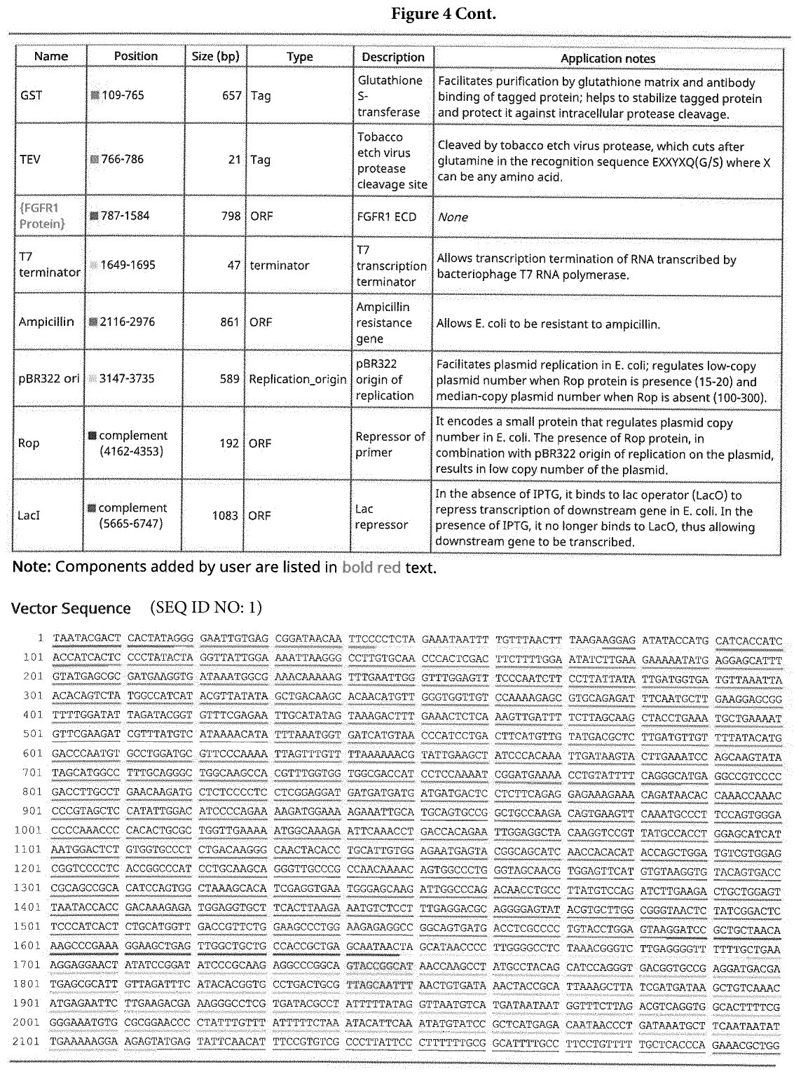

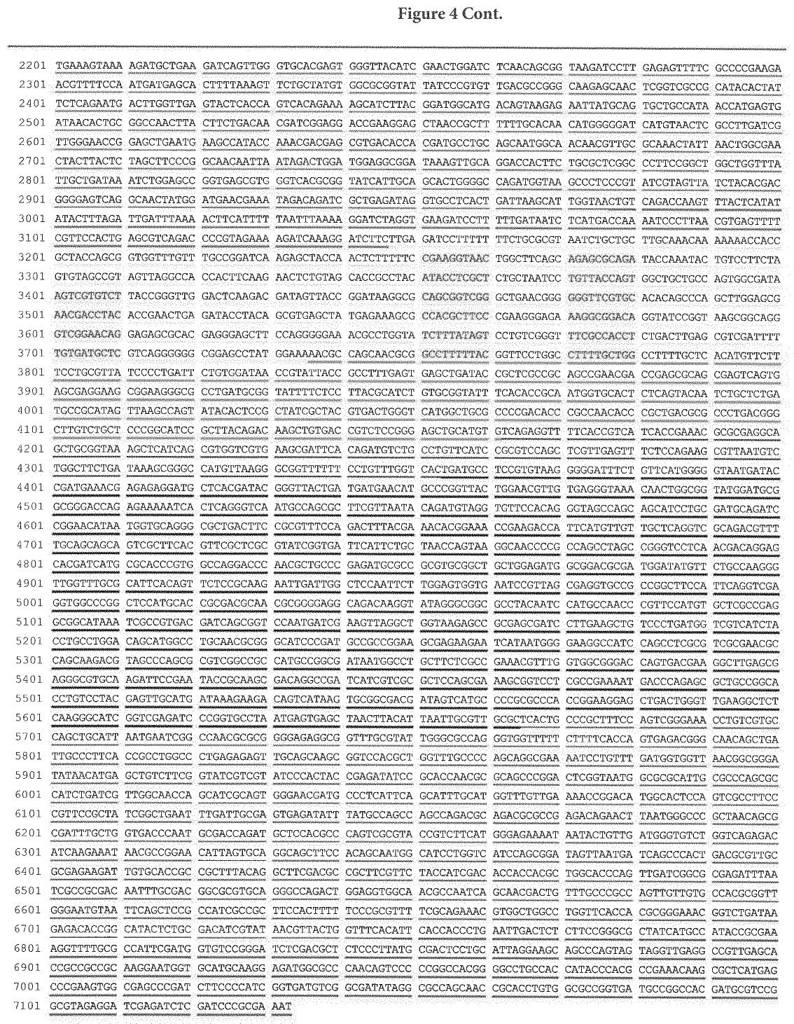

Although any number of sources are available for human FGFR1 for use in activating or pre-treating the NK cells, or in particular GDT cells, as described herein, an option is to express and purify the extracellular portion of human FGFR1 from a plasmid using the expression construct as shown in FIG. 4. This is a non-limiting example and any source of FGFR1 can be used.

The terms “co-administration” or the like, are meant to encompass administration of the selected therapeutic agents to a single patient, and are intended to include treatment regimens in which the agents are administered by the same or different route of administration or at the same or different time.

As used herein, the term “agonist” refers to a compound, the presence of which results in a biological activity of a protein that is the same as the biological activity resulting from the presence of a naturally occurring ligand for the protein.

As used herein, the term “partial agonist” refers to a compound the presence of which results in a biological activity of a protein that is of the same type as that resulting from the presence of a naturally occurring ligand for the protein, but of a lower magnitude.

As used herein, the term “antagonist” refers to a compound, the presence of which results in a decrease in the magnitude of a biological activity of a protein. In certain embodiments, the presence of an antagonist results in complete inhibition of a biological activity of a protein. In certain embodiments, an antagonist is an inhibitor.

“Administering” when used in conjunction with a therapeutic composition (e.g. activated NK cells, or the combination of activated cells) means to administer a therapeutic directly into or onto a target tissue or to administer a therapeutic to a patient whereby the therapeutic positively impacts the tissue to which it is targeted.

The CD3 protein is composed of three pairs of dimers (εδ, ζζ) that are responsible for intracellular signaling, initiated by the phosphorylation of immunoreceptor tyrosine activation motifs (ITAMs) (ten in total). CD3 is initially expressed in the cytoplasm of pro-thymocytes, the stem cells from which T-cells arise in the thymus. The pro-thymocytes differentiate into common thymocytes, and then into medullary thymocytes, and it is at this latter stage that CD3 antigen begins to migrate to the cell membrane. The antigen is found bound to the membranes of all mature T cells, and in virtually no other cell type, although it does appear to be present in small amounts in Purkinje cells. This high specificity, combined with the presence of CD3 at all stages of T cell development, makes it a useful immunohistochemical marker for T cells in tissue sections. The antigen remains present in almost all T-cell lymphomas and leukemias, and can therefore be used to distinguish them from superficially similar B-cell and myeloid neoplasms.

The term “subject” or “patient” as used herein includes, but is not limited to, humans and non-human vertebrates such as wild, domestic, and farm animals. The terms can be used interchangeably. In certain embodiments, the subject or patient described herein is an animal. In certain embodiments, the subject or patient is a mammal. In certain embodiments, the subject is a human. In certain embodiments, the subject or patient is a non-human animal. In certain embodiments, the subject or patient is a non-human mammal. In certain embodiments, the subject or patient is a domesticated animal, such as a dog, cat, cow, pig, horse, sheep, or goat. In certain embodiments, the subject or patient is a companion animal such as a dog or cat. In certain embodiments, the subject or patient is a livestock animal such as a cow, pig, horse, sheep, or goat. In certain embodiments, the subject or patient is a zoo animal. In another embodiment, the subject or patient is a research animal such as a rodent, dog, or non-human primate. In certain embodiments, the subject or patient is a non-human transgenic animal such as a transgenic mouse or transgenic pig.

The term “inhibit” includes the administration of a therapeutic of embodiments herein to prevent the onset of the symptoms, alleviating the symptoms, or eliminating the disease, condition or disorder.

By “pharmaceutically acceptable”, it is meant the carrier, diluent or excipient must be compatible with the other ingredients of the therapeutic and not deleterious to the recipient thereof.

The terms “treat,” “treated,” or “treating” as used herein refers to both therapeutic treatment and prophylactic or preventative measures, wherein the object is to inhibit, prevent or slow down (lessen) an undesired physiological condition, disorder or disease, or to improve, inhibit, or otherwise obtain beneficial or desired clinical results. For the purposes of this invention, beneficial or desired clinical results include, but are not limited to, improvement or alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

“Activated NK cell”, as used herein, refers to the state of an NK cell that has been sufficiently stimulated to be considered a cytotoxic cell, versus a regulatory cell. Activation can also be associated with induced cytokine production, and detectable effector functions. The term “activated NK cells” refers to, among other things, NK cells that are CD56dimCD16+, CD56int/lowCD16−, CD56+CD16+ cytotoxic cells and/or ADCCs. In some embodiments, the NK cells are activated as provided for herein.

### Chemotherapeutic or Lymphosuppressive Agents and Immunosuppressive Agents

Lymphosuppressant or lymphosuppressive agent as used herein in pre-conditioning steps can include steroid or other immunosuppressive agents such as prednisone or a monoclonal antibody against CD20, e.g., rituximab. The typical dose of an immunosuppressive agent is between 25-1000 mg/m², for the desired preconditioning time-frames, outlined in Tables 1-5.

Pre-conditioning agents can also include an effective amount (typically a low dose) of any one or combination of a chemotherapeutic or antineoplastic agent such as thioguanine (6TG), 6-mercaptopurine (6-MP), and purine analogues such as clofarabine, fludarabine, cytarabine. The typical dose of these agents is between 25-1000 mg/m², for the desired time-frame of 3, or 4, or 5 days in advance of administering the activated cellular composition.

In certain embodiments, the patient can be pre-conditioned with a lymphosuppressive or chemotherapeutic agent such as cyclophosphamide with or without fludarabine. Examples of such pre-conditioning treatments can be found for example in U.S. Pat. No. 9,855,298. A non-limiting example is administering fludarabine (30 mg/m² intravenous daily for 4 days) and cyclophosphamide (500 mg/m² intravenous daily for 2 days starting with the first dose of fludarabine). After administration, the activated lymphocytic compositions can be administered 2 to 5 days after completion of the fludarabine (i.e., following the pre-conditioning treatment). In certain embodiments, the cyclophosphamide is administered for 2-3 days at a dose of about 500 to about 600 mg/m²).

Activating Cytokines, Suitable for Activating Nk Cells as well as GDT Cells and iNK Cells

Suitable NK activating cytokines can include an effective amount of one or more of the following: IL-2, IL-15, IL-21, Flt3-L, SCF, IL-7, IL-12, and IL18, and any combination thereof. These activating cytokines are also effective as single agents, or in combination for activating NK cells, iNKT cells, and GDT cells.

### Nonsteroidal Anti-Inflammatory Drugs (NSAIDs) for Post Infusion, Post-conditioning Agents

NSAIDs work by reducing the production of prostaglandins, chemicals that promote inflammation, pain, and fever. Suitable NSAIDs for controlling negative aspects of the immune response in the methods described herein, include specific COX-2 inhibitors such as celecoxib (Celebrex) as well as non-specific inhibitors such as aspirin, as well as non-specific COX-2 inhibitors such as: indomethacin (Indocin), ibuprofen (Advil, Motrin), naproxen (Naprosyn), piroxicam (Feldene), and nabumetone (Relafen).

Prostaglandins are made by two different enzymes, cyclooxygenase-1 (COX-1) and cyclooxygenase-2 (COX-2). The prostaglandins made by the two different enzymes have slightly different effects on the body. Prostaglandin E2 (PGE2) is a terminal prostaglandin in the cyclooxygenase (COX) pathway. Inhibition of PGE2 production may relieve inflammatory symptoms such as fever, arthritis, and inflammatory pain. COX-2 inhibitors are a subclass of NSAIDs that selectively block the COX-2 enzyme and not the COX-1 enzyme. Blocking this enzyme impedes the production of prostaglandins by the COX-2 which is more often the cause the pain and swelling of inflammation and other painful conditions. Because they selectively block the COX-2 enzyme and not the COX-1 enzyme, these drugs are uniquely different from traditional NSAIDs which usually block both COX-1 and COX-2 enzymes. COX-1 is an enzyme which is normally present in a variety of tissues in the body, including sites of inflammation and the stomach. Some of the prostaglandins made by COX-1 protect the inner lining of the stomach.

Common NSAIDs such as aspirin block both COX-1 and COX-2. When the COX-1 enzyme is blocked, inflammation is reduced, but the protection of the lining of the stomach also is lost. This can cause stomach upset as well as ulceration and bleeding from the stomach and even the intestines.

The COX-2 enzyme is located specifically in areas of the body that commonly are involved in inflammation but not in the stomach. When the COX-2 enzyme is blocked, inflammation is reduced; however, since the COX-2 enzyme does not play a role in protecting the stomach or intestine, COX-2 specific NSAIDs do not have the same risk of injuring the stomach or intestines.

Older NSAIDs (for example, aspirin, ibuprofen, naproxen, etc.) all act by blocking the action of both the COX-1 and COX-2 enzymes. COX-2 inhibitors selectively block the COX-2 enzyme and therefore have a lower risk of causing ulcers of the stomach or intestine.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. The antibodies may exist in a variety of forms including, for example, polyclonal antibodies, monoclonal antibodies, Fv, Fab and F(ab)2, as well as single chain antibodies and humanized antibodies.

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the embodiments include, but are not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

Cancers that may be treated include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the activated NK cells described herein include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyo sarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

The term “anti-tumor effect” as used herein, refers to a biological effect that can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies to prevent the occurrence of tumor in the first place.

The term “auto-antigen” means any self-antigen which is mistakenly recognized by the immune system as being foreign. Auto-antigens comprise, but are not limited to, cellular proteins, phosphoproteins, cellular surface proteins, cellular lipids, nucleic acids, glycoproteins, including cell surface receptors.

As used herein, the term “autologous” is meant to refer to any material derived from the same individual to which it is later to be re-introduced into the individual.

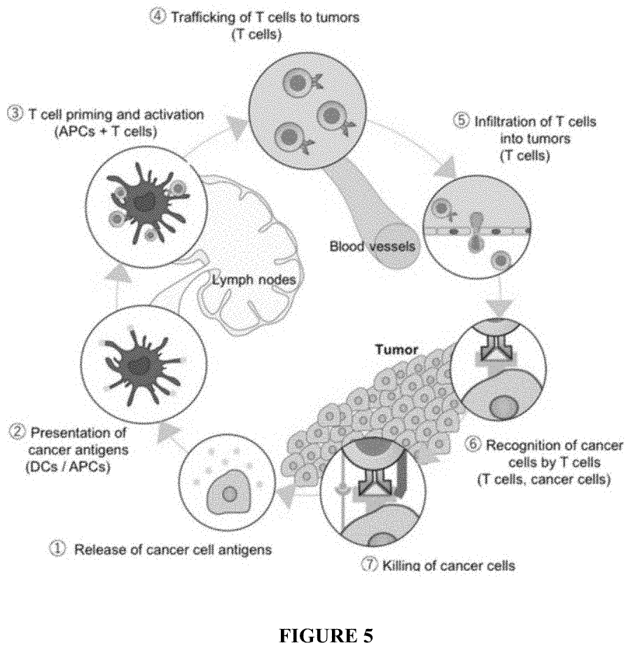

“Allogeneic” refers to a graft derived from a different animal of the same species. For purposes of the present treatments, the activated NK cells are completely HLA mismatched, unlike the situations in renal and bone marrow transplantations where matching HLA-A, -B, and -DR are beneficial for graft survival. In the present methods for treating cancer, infectious conditions including HIV, the activated NK cells target the cancer cells and bind to them, and kill them, releasing tumor cell antigens. This cycle repeats itself with the allogeneic activated NK cells, until they are killed by the host cell immune responses within about 4-7 days (and possibly during an extended range of 4-15 days). Thus, there is no danger for GVHD with such a short life-span for the allogeneic cells. However, in the process of recognizing and killing tumor cells and releasing antigens for that span of about 4-7 days, the allogeneic cells can serve to vaccinate the host NK and other immune cells to recognize and kill the tumor cells. See additionally, review article Cancer Sci. 2019 January; 110(1):16-22. FIG. 5 is a diagram showing the cancer immunity cycle. Cancer-immunity cycle. The induction of antitumor immunity is a cyclic process that can be self-propagating. It can amplify and extend T cell responses against cancer cells. It also contains several inhibitory factors itself to halt the cycle when the target cells (cancer cells) are eradicated. The cycle can be divided into 7 steps, as shown in FIG. 5, starting with the release of cancer antigens from the cancer cells and ending with the killing of cancer cells. APC, antigen-presenting cell; DC, dendritic cell.

“Xenogeneic” refers to a graft derived from an animal of a different species.

The present invention relates to a method for treating cancer and infectious conditions, including HIV, comprising administering activated lymphocytic cells, including essentially pure activated natural killer cells, which are activated in the presence of cytokines, and optionally soluble FGFR1.

Therefore, according to one aspect of the present invention, a method for activating natural killer cells isolated from various sources, including peripheral blood mononuclear cells is provided, in the presence of cytokines, in combination with the pre-conditioning and post-conditioning regimens described herein.

“Peripheral blood mononuclear cells,” “PBMCs” or “mononuclear cells” refer to mononuclear cells separated from peripheral blood typically used for anti-cancer immunotherapy. The peripheral blood mononuclear cells can be obtained from human blood collected using known methods such as the Ficoll-Hypaque density gradient method.

According to one exemplary embodiment of the present invention, “peripheral blood mononuclear cells” may be obtained from any suitable person. The source of the donor lymphocytic cells, including sources such as peripheral blood mononuclear cells, as used herein are required to be allogeneic to the recipient patient for isolation of the desired lymphocytic cells including: NK cells, γδT cells, iNKT cells, and CD3 T cells for use in the anti-cancer, anti-viral, and/or anti-bacterial immunotherapy methods according to the present invention. Thus, the donor inhibitory ligand mismatches with the patient (e.g. host) HLA.

### Exemplary Activating Cytokines and Lymphocytic Cell and NK Activation

In the present invention, the term “cytokine” refers to an immune activating cytokine that can be used to activate any of the therapeutic lymphocytic cells described herein, for use in the present methods, including the essentially pure NK cells, and including those isolated from peripheral blood mononuclear cells. According to one exemplary embodiment of the present invention, IL-2, IL-15, IL-21, Flt3-L, SCF, IL-7, IL-12, or IL18 may be used as such a cytokine alone or in combination. In particular, since IL-2, IL-15 or IL-21 is known as a cytokine having an excellent effect in differentiation of the peripheral blood mononuclear cells into the NK cells and proliferation of the NK cells, it is desirable to use one or a combination of these cytokines. According to one exemplary embodiment of the present invention, IL-2 is used, but the present invention is not limited thereto. Additionally included in embodiments of the present methods are IL-2 agonists, IL-2 homologs, or other synthetic, optimized IL-2 compositions. (See U.S. Pat. NO. 6,921,530 describing low dose IL-2 for potentiation of immunity).

The IL-15 plays a part in differentiation into NK cells. This is found from the fact that the NK cells are lacking in the mice which lack transcription factor interferon (IFN)-regulatory factor 1 required for IL-15 production (Kouetsu et al., Nature 391,700-703, 1998), and that the NK cells are not found in the mice in which IL-15 or IL-15Ra is lacking. As a result, it has been reported that IL-15 directly promotes growth and differentiation of the NK cells by means of the IL-15 receptor expressed in the NK cells (MrozekE et al., Blood 87, 2632-2640, 1996).

IL-21 is a cytokine secreted by activated CD4+ T cells (Nature, 5:688-697, 2005), and the IL-21 receptor (IL-21R) is expressed in lymphocytes such as dendritic cells, NK cells, T cells, and B cells (Rayna Takaki, et al., J. Immonol 175: 2167-2173, 2005). IL-21 is structurally highly similar to IL-2 and IL-15, and IL-21R shares a chain with IL-2R, IL-15, IL-7R, and IL-4R (Asao et al., J. Immunol, 167: 1-5, 2001). IL-21 has been reported to induce maturation of an NK cell precursor from bone marrow (Parrish-Novak, et al., Nature, 408: 57-63, 2000), particularly promote effector functions such as an ability of the NK cells to produce cytokines and kill cells (M. Strengell, et al., J Immunol, 170, 5464-5469, 2003; J. Brady, et al., J Immunol, 172, 2048-2058, 2004), and promote the anti-cancer response of the innate and adaptive immune systems by enhancing the effector functions of CD8+ T cells (Rayna Takaki, et al., J Immunol 175, 2167-2173, 2005; A. Moroz, et al., J Immunol, 173, 900-909, 2004). Also, IL-21 has been reported to activate the NK cells separated from human peripheral blood (Parrish-Novak, et al., Nature, 408, 57, 2000), and induce mature NK cells from hematopoietic stem cells separated from cord blood (J. Brady, et al., J Immunol, 172, 2048, 2004).

Embodiments of the present invention include using the one or more of these “activating cytokines” at a concentration of 50 U/ml to 1,000 U/ml, for example, 200 U/ml to 800 U/ml, or 400 U/ml to 600 U/ml to activate any of the lymphocytic cells such as NK cells, gamma delta T cells, or iNKT cells described herein for use in the present immunomodulatory methods.

A “disease” is a state of health of a subject wherein the subject cannot maintain homeostasis, and wherein if the disease is not ameliorated then the animal's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared X 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. An “isolated” biological component (such as a nucleic acid, protein or cell) has been substantially separated or purified away from other biological components (such as cell debris, other proteins, nucleic acids or cell types). Biological components that have been “isolated” include those components purified by standard purification methods.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Chemotherapy includes treatment with a chemical agent (such as a cytotoxic agent) with therapeutic utility for treating diseases characterized by abnormal cell growth, such as tumors, neoplasms, cancer and psoriasis.

As used herein, recombinant generally refers to the following: A recombinant nucleic acid or protein is one that has a sequence that is not naturally occurring or has a sequence that is made by an artificial combination of two otherwise separated segments of sequence. This artificial combination is often accomplished by chemical synthesis or by the artificial manipulation of isolated segments of nucleic acids, for example, by genetic engineering techniques.

As used herein, the following abbreviations for the commonly occurring nucleic acid bases are used. “A” refers to adenosine, “C” refers to cytosine, “G” refers to guanosine, “T” refers to thymidine, and “U” refers to uridine.

The term “leukocytes” or “white blood cell” as used herein refers to any immune cell, including monocytes, neutrophils, eosinophils, basophils, and lymphocytes. The term “lymphocytes” as used herein refer to cells commonly found in lymph, and include natural killer cells (NK cells), T-cells, and B-cells. It will be appreciated by one of skill in the art that the above listed immune cell types can be divided into further subsets.

The term “tumor infiltrating leukocytes” as used herein refers to leukocytes that are present in a solid tumor.

The term “blood sample” as used herein refers to any sample prepared from blood, such as plasma, blood cells isolated from blood, and so forth.

The term “purified sample” as used herein refers to any sample in which one or more cell subsets are enriched. A sample may be purified by the removal or isolation of cells based on characteristics such as size, protein expression, and so forth.

Pharmaceutically acceptable vehicles: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compositions, and additional pharmaceutical agents.

In general, the nature of a suitable carrier or vehicle for delivery will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

In some embodiments, compositions, whether they be solutions, suspensions or other like form, may include one or more of the following: DMSO, sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

Diseases that the compositions and methods described herein can treat include microbial infections such as a viral infection, yeast infection, fungal infection, protozoan infection and/or bacterial infection.

Exemplary bacterial infections include those caused by one or more of the pathogenic bacterial species Bacteroides, Clostridium, Streptococcus, Staphylococcus, Pseudomonas, Haemophilus, Legionella, Mycobacterium, Escherichia, Salmonella, Shigella, or Listeria.

By a “viral infection” is meant an infection caused by the presence of a virus in the body. Viral infections include chronic or persistent viral infections, which are viral infections that are able to infect a host and reproduce within the cells of a host over a prolonged period of time-usually weeks, months or years, before proving fatal. Viruses giving rise to chronic infections that which may be treated in accordance with the present invention include, for example, the human papilloma viruses (HPV), Herpes simplex, and other herpes viruses, the viruses of hepatitis B and C as well as other hepatitis viruses, human immunodeficiency virus, and the measles virus, all of which can produce important clinical diseases. Prolonged infection may ultimately lead to the induction of disease which may be, e.g., in the case of hepatitis C virus liver cancer, fatal to the patient. Other chronic viral infections which may be treated in accordance with the present invention include Epstein Barr virus (EBV), as well as other viruses such as those which may be associated with tumors.

Examples of viral infections which can be treated or prevented with the activated NK cell compositions and methods described herein include, but are limited to, viral infections caused by retroviruses (e.g., human T-cell lymphotrophic virus (HTLV) types I and II and human immunodeficiency virus (HIV)), herpes viruses (e.g., herpes simplex virus (HSV) types I and II, Epstein-Ban virus and cytomegalovirus), arenaviruses (e.g., lassa fever virus), paramyxoviruses (e.g., morbillivirus virus, human respiratory syncytial virus, and pneumovirus), adenoviruses, bunyaviruses (e.g., hantavirus), coronaviruses, filoviruses (e.g., Ebola virus), flaviviruses (e.g., hepatitis C virus (HCV), yellow fever virus, and Japanese encephalitis virus), hepadnaviruses (e.g., hepatitis B viruses (HBV)), orthomyoviruses (e.g., Sendai virus and influenza viruses A, B and C), papovaviruses (e.g., papillomaviruses), picornaviruses (e.g., rhinoviruses, enteroviruses and hepatitis A viruses), poxviruses, reoviruses (e.g., rotaviruses), togaviruses (e.g., rubella virus), and rhabdoviruses (e.g., rabies virus). The treatment and/or prevention of a viral infection includes, but is not limited to, alleviating one or more symptoms associated with said infection, the inhibition, reduction or suppression of viral replication and/or viral load, and/or the enhancement of the immune response.

As used herein, immunodeficient means lacking in at least one essential function of the immune system. As used herein, an “immunodeficient” subject (such as a human) is one lacking specific components of the immune system or lacking function of specific components of the immune system (such as, for example, B cells, T cells, NK cells or macrophages). In some cases, an immunodeficient subject comprises one or more genetic alterations that prevent or inhibit the development of functional immune cells (such as B cells, T cells or NK cells). In some examples, the genetic alteration is in IL17 or IL17 receptor.

As used herein, immunosuppressed refers to a reduced activity or function of the immune system. A subject can be immunosuppressed, for example, due to treatment with an immunosuppressant compound or as a result of a disease or disorder (for example, immunosuppression that results from HIV infection or due to a genetic defect). In some cases, immunosuppression occurs as the result of a genetic mutation that prevents or inhibits the development of functional immune cells, such as T cells.

As used herein, interleukin 8 (IL8) is a member of the CXC chemokine family that is a major mediator of the inflammatory response. IL8 is secreted by several cell types and functions as a chemoattractant and potent angiogenic factor. Human IL8 is a functional equivalent of mouse CXCL1 and CXCL2. Nucleotide and amino acid sequences for IL8 are publically available. For example, human IL8 sequences can be found under NCBI Gene ID 3576.

As used herein, interleukin 17A (IL17A) is a proinflammatory cytokine produced by activated T cells. Nucleotide and amino acid sequences for IL17A are publically available. For example, human and mouse IL17A sequences can be found under NCBI Gene ID 3605 and Gene ID 16171, respectively.

In some embodiments of the invention, a “therapeutically effective amount” is an amount of an activated cellular composition described herein that results in a reduction in viral titer or microbial titer by at least 2.5%, at least 5%, at least 10%, at least 15%, at least 25%, at least 35%, at least 45%, at least 50%, at least 75%, at least 85%, by at least 90%, at least 95%, or at least 99% in a subject/patient/animal administered an activated cellular composition and treated with a related method described herein, relative to the viral titer or microbial titer in an animal or group of animals (e.g., two, three, five, ten or more animals) not administered a composition of the invention.

Examples of types of cancer and proliferative disorders that can be treated with an effective amount of the activated lymphocytic cellular compositions and related methods described herein include, but are not limited to, leukemia (e.g., myeloblastic, promyelocytic, myelomonocytic, monocytic, erythroleukemia, chronic myelocytic (granulocytic) leukemia, and chronic lymphocytic leukemia), lymphoma (e.g., Hodgkin's disease and non-Hodgkin's disease), fibro sarcoma, myxo sarcoma, lipo sarcoma, chondro sarcoma, osteogenic sarcoma, angio sarcoma, endotheliosarcoma, Ewing's tumor, colon carcinoma, pancreatic cancer, breast cancer, ovarian cancer, prostate cancer, squamous cell carcinoma, basal cell carcinoma, adenocarcinoma, renal cell carcinoma, hepatoma, Wilm's tumor, cervical cancer, uterine cancer, testicular tumor, lung carcinoma, small cell lung carcinoma, bladder carcinoma, epithelial carcinoma, glioma, astrocytoma, oligodendroglioma, melanoma, neuroblastoma, retinoblastoma, dysplasia and hyperplasia. The treatment and/or prevention of cancer includes, but is not limited to, alleviating one or more symptoms associated with cancer, the inhibition or reduction of the progression of cancer, the promotion of the regression of cancer, and/or the promotion of the immune response.

In certain other embodiments, a “therapeutically effective amount” is the amount of the activated lymphocytic cellular composition that results in a reduction of the growth or spread of cancer by at least 2.5%, at least 5%, at least 10%, at least 15%, at least 25%, at least 35%, at least 45%, at least 50%, at least 75%, at least 85%, by at least 90%, at least 95%, or at least 99% in a patient or an animal administered a composition described herein relative to the growth or spread of cancer in a patient (or an animal) or a group of patients (or animals) not administered a composition of the invention.

In some embodiments, the activated lymphocytic cellular compositions are used in a method of treating a yeast or bacterial infection. For example, the compositions and methods described herein can treat infections relating to Streptococcus pyogenes, Streptococcus pneumoniae, Neisseria gonorrhoea, Neisseria meningitidis, Corynebacterium diphtheriae, Clostridium botulinum, Clostridium perfringens, Clostridium tetani, Haemophilus influenzae, Klebsiella pneumoniae, Klebsiella ozaenae, Klebsiella rhinoscleromotis, Staphylococcus aureus, Vibrio cholera, Escherichia coli, Pseudomonas aeruginosa, Campylobacter (Vibrio) fetus, Campylobacter jejuni, Aeromonas hydrophila, Bacillus cereus, Edwardsiella tarda, Yersinia enterocolitica, Yersinia pestis, Yersinia pseudotuberculosis, Shigella dysenteriae, Shigella flexneri, Shigella sonnei, Salmonella typhimurium, Treponema pallidum, Treponema pertenue, Treponema carateneum, Borrelia vincentii, Borrelia burgdorferi, Leptospira icterohemorrhagiae, Mycobacterium tuberculosis, Toxoplasma gondii, Pneumocystis carinii, Francisella tularensis, Brucella aborts, Brucella suis, Brucella melitensis, Mycoplasma spp., Rickettsiaprowazeki, Rickettsia tsutsugumushi, Chlamydia spp., Helicobacter pylori or any combination thereof.

In certain embodiments, the activated lymphocytic cellular compositions can be administered simultaneously with anti-microbial, anti-viral and other therapeutic agents. Alternatively, activated lymphocytic cellular compositions can be administered at selected times in advance of times when anti-microbial, anti-viral and other therapeutic agents are administered.

In some embodiments, methods of treating a patient with HIV, cancer, a viral infection, or a bacterial infection are provided. In some embodiments, the methods comprise administering an effective amount of a lymphocytic cellular composition comprising activated natural killer (NK) cells to the patient. In some embodiments, the patient has HIV. In some embodiments, the patient has cancer. In some embodiments, the patient has a viral infection. In some embodiments, the patient has a bacterial infection. Non-limiting examples of cancer, viral infections and bacterial infections are provided for herein.

In some embodiments, methods for increasing an immune response in a patient in need thereof are provided. In some embodiments, the methods comprise administering an effective amount of a lymphocytic cellular composition comprising activated NK cells to the patient.

In some embodiments, methods of inhibiting HIV replication or reproduction in a patient are provided. In some embodiments, the methods comprise administering to the subject with HIV an effective amount of a lymphocytic cellular composition comprising activated natural killer (NK) cells to the patient. As used herein, the term “inhibiting HIV replication or reproduction” can refer to the virus being unable to replicate or reproduce in the subject to maintain a detectable viral load. In some embodiments, the method comprises reducing the HIV viral load of a subject infected with HIV comprising administering to the subject with HIV an effective amount of a lymphocytic cellular composition comprising activated natural killer (NK) cells to the patient. The viral load can be reduced to undetectable levels or by at least 10, 20, 30, 40, 50, 60, 70, 80, 90, 91, 92, 93, 94, 95, 96, 96, 97, 98, or 99% as compared to the pre-treatment levels.

In some embodiments, methods of inhibiting tumor growth in a patient are provided. In some embodiments, the methods comprise administering to the patient with the tumor an effective amount of a lymphocytic cellular composition comprising activated natural killer (NK) cells to the patient. The tumor growth can be slowed or eliminated. In some embodiments, the tumor is put into remission, in that it is no longer detectable. In some embodiments, the metastases of a tumor is inhibited.

In some embodiments, methods of inhibiting a viral or bacterial replication or reproduction in a subject having a viral or bacterial infection are provided. In some embodiments, the methods comprise administering to the subject with the viral or bacterial infection an effective amount of a lymphocytic cellular composition comprising activated natural killer (NK) cells to the patient. In some embodiments the viral or bacterial load in the patient is reduced or is undetectable. The viral or bacterial load can be reduced to undetectable levels or by at least 10, 20, 30, 40, 50, 60, 70, 80, 90, 91, 92, 93, 94, 95, 96, 96, 97, 98, or 99% as compared to the pre-treatment levels.

As provided herein, in some embodiments, the cellular composition can also comprise activated gamma delta T cells (GDT cells). The GDT cells can also be administered in a separate composition from the NK cells.

As provided herein, in some embodiments, the cellular composition can also comprise invariant natural killer T cells (iNKT cells). The iNKT cells can also be administered in a separate composition from the NK cells and/or the GDT cells.

As provided herein, in some embodiments, the cellular composition can also comprise invariant CD3+ T cells. The CD3+ T cells can also be administered in a separate composition from the NK cells, the GDT cells, and/or the iNKT cells.

In some embodiments, the cells are activated by mixing/incubating/contacting the cells with at least one cytokine and optionally soluble fibroblast growth factor receptor 1 (sFGFR1). This mixing/incubating/contacting can be done in vitro or outside the patient, which can also be referred to as ex vivo. In some embodiments, the cells are not activated, or initially activated, by the administration of an activating agent to the subject. In some embodiments, the cell's activation can be maintained or enhanced by the exogenous administration of a cytokine to the subject after the activated cells are administered to the subject. In some embodiments, the cytokine is selected from the group consisting of IL-2, IL-15, IL-21, Flt3-L, stem cell factor (SCF), IL-7, IL-12, and IL-18, and any combination thereof. In some embodiments, the cytokine is IL-2. In some embodiments, the cytokine is IL-15. In some embodiments, the cytokine is IL-21. In some embodiments, the cytokine is Flt3-L. In some embodiments, the cytokine is stem cell factor (SCF). In some embodiments, the cytokine is IL-7. In some embodiments, the cytokine is IL-12. In some embodiments, the cytokine is IL-18. In some embodiments, the cytokine is incubated/mixed/contacted with the cellular composition ex vivo for between about 6-24 hours. In some embodiments, the amount of cytokine incubated/mixed/contacted with the cellular composition ex vivo is from about 100-1000 IU/ml. In some embodiments, the subject is pre-conditioned prior to administering the lymphocytic cellular composition to the subject. Without being bound to any particular theory, in some embodiments, the pre-conditioning lymphodepletes the subject's own immune cells and creates room or space for the new immune cells that are then subsequently going to be administered to the patient. In some embodiments, the pre-conditioning comprises administering at least one lympho-suppressive agent, chemotherapeutic agent, or immunosuppressive agent for about 1 to about 10 days, 3 to about 7 days, about 1 to about 2 days prior to administering the lymphocytic cellular composition. In some embodiments, the pre-conditioning comprises administering fludarabine, cyclophosphamide, and/or interferon alpha. The method of claim 13, wherein the lympho-suppressive or chemotherapeutic agent comprises any one of 6TG, 6-MMP, one or more purine analogues selected from the group consisting of clofarabine, fludarabine, and cytarabine, or any combination thereof. In some embodiments, the dose of the lympho-suppressive or chemotherapeutic agent ranges from about 5 mg/m² to about 50 mg/m². In some embodiments, the chemotherapeutic or immunosuppressive agent comprises any cyclophosphamide, rituximab, a steroid, or any combination thereof. In some embodiments, the steroid is a glucocorticoid steroid. In some embodiments, the steroid is prednisone. In some embodiments, the pre-conditioning comprises administering fludarabine from day −7 to day −3. In some embodiments, the fludarabine is administered at a dose of about 15 mg/m²/d. In some embodiments, the pre-conditioning comprises administering cyclophosphamide on day −2 and day −1. In some embodiments, the dose of the cyclophosphamide is as provided herein. In some embodiments, the pre-conditioning comprises administering, interferon alpha on day −2 and day −1. In some embodiments the dose of the interferon alpha is about 3×10⁶ IU/m²/d. As used herein, the term “day −1” refers to days prior to the administration of the activated cellular composition. For example, Day 0 is considered the day that the cellular composition comprising the NK cells are administered to the subject. Therefore, “Day −1” refers to the day before the administration. “Day −2” would refer to two days before administration and so on and so forth. In some embodiments, the method comprising administering on Day 0 the activated NK cells, which can be part of the cellular composition. In some embodiments, the activated GDT cells are also administered. They can be administered as part of the same composition or separately. Additionally, in some embodiments, the patient is also administered IL-2 on Day 0. The IL-2 can be, for example, administered intravenously or subcutaneously. In some embodiments, the IL-2 is administered for 1 to about 5 days after administration of the activated cellular composition, such as on Day +1, Day +2, Day +3, Day +4, and Day +5. The IL-2 could be administered longer as well. The term “Day +1” and the like refers to the days after administration of the activated cellular composition. In some embodiments, the IL-2 is administered at a dose of about 6×10⁶ IU/m². In some embodiments, the IL-2 is not administered to the subject after Day 0.

In some embodiments, the subject is administered with an anti-inflammatory, such as a nonsteroidal anti-inflammatory drug (NSAID). These can be given for about 1 to about 60 days after Day 0. In some embodiments, the NSAID is a Cox-2 inhibitor, such as those provided herein. In some embodiments, the NSAID is celecoxib. In some embodiments, the NSAID is ibuprofen or naproxen. In some embodiments, the NSAID is administered for about 14 to about 60 days following administration of the lymphocytic cellular composition.

Also provided herein are methods of activating NK cells, the method comprising contacting a cellular composition comprising the NK cells in vitro with at least one cytokine and/or soluble fibroblast growth factor receptor 1 (sFGFR1). In some embodiments, the cytokine is IL-2. In some embodiments, the cytokine is selected from the group consisting of IL-2, IL-15, IL-21, Flt3-L, stem cell factor (SCF), IL-7, IL-12, and IL18, and any combination thereof. In some embodiments, the cytokine is contacted with the cellular composition for from about 6 to about 24 hours. In some embodiments, the amount of cytokine contacted with the cellular composition is from about 100 IU/ml to about 1000 IU/ml. In some embodiments, the composition can also comprise gamma delta T cells (GDT cells), invariant natural killer T cells (iNKT cells), and/or CD3+ T cells. The cells can be isolated from any source. For example, in some embodiments, the NK cells, gamma delta T cells (GDT cells), invariant natural killer T cells (iNKT cells), and/or CD3+ T cells are isolated from peripheral blood. They can be isolated through, for example, leukapheresis.

Also provided herein are cytokine and/or soluble fibroblast growth factor receptor 1 (sFGFR1) activated lymphocytic cellular composition comprising activated natural killer (NK) cells. In some embodiments, the activated composition is an IL-2 activated composition. In some embodiments, the cytokine activated composition is a IL-2, IL-15, IL-21, Flt3-L, stem cell factor (SCF), IL-7, IL-12, and IL18, and any combination thereof, activated composition. In some embodiments, the activated composition comprises gamma delta T cells (GDT cells), invariant natural killer T cells (iNKT cells), and/or CD3+ T cells. In some embodiments, the NK cells are peripheral blood NK cells. In some embodiments, the gamma delta T cells (GDT cells), invariant natural killer T cells (iNKT cells), CD3+ T cells are peripheral blood gamma delta T cells (GDT cells), invariant natural killer T cells (iNKT cells), and CD3+ T cells.

Combinations with Antibodies

For example, the activated lymphocytic cellular compositions can be administered simultaneously with antibodies specific for a selected cancer type. Alternatively, activated lymphocytic cellular compositions can be administered at selected times in advance of times when antibodies specific for a selected cancer type are administered. Antibodies specific for a selected cancer type include any antibody approved for treatment of cancer. Examples include trastuzumab (Herceptin) for breast cancer, rituximab (Rituxan) for lymphoma, and cetuximab (Erbitux) for head and neck squamous cell carcinoma.

Additional examples of such antibody agents include inhibitors of PD-1 or PD-L1 (B7-H1), such as anti-PD-1 antibodies, including nivolumab (Nivolumab from Bristol-Myers Squibb) and pembrolizumab/lambrolizumab, also known as MK-3475 (Keytruda from Merck), pidilizumab (Curetech), AMP-224 (Amplimmune), and anti-PD-L1 antibodies, including MPDL3280A (Roche), MDX-1105 (Bristol Myer Squibb), MEDI-4736 (Astra7eneca) and MSB-0010718 C (Merck). Other checkpoint inhibitors include antagonists of CTLA-4, such as anti-CTLA-4 antibodies. An exemplary anti-CTLA4 antibody is Yervoy™ (ipilimumab) marketed by Bristol-Myers Squibb. Other exemplary CTLA-4 antibodies include tremelimumab (Pfizer), Ticilimumab (AstraZeneca) and AMGP-224 (Glaxo Smith Kline). An exemplary treatment regimen using an immunotherapeutic antibody can be found in Example 3. Combinations with any two of these antibodies may also be indicated in certain instances. The pre-conditioning and post-conditioning regimens, as described in FIG. 1, along with the different cellular infusion options can be applied to this antibody treatment (i.e., the antibody is administered at a time following the cellular infusion, from 4-14 days following the infusion, preferably 4-6 days following the infusion).

### Sources of Allogeneic NK Cells Used for Therapeutic Lymphocytic Compositions

Commonly used allogeneic NK cells are apheresis products collected from haploidentical and unrelated donor PBMC (Koepsell et al. Transfusion (2013) 53(2):404-10).

Another source is umbilical cord blood (UCB), where NK cells are generated from CD34+ progenitor cells that undergo expansion and differentiation using cytokines and growth factors and thereby mature into cytolytic NK cells (Spanholtz J. et al. PLoS One (2011) 6(6):e20740; and Arai S. et al. Cytotherapy (2008) 10(6):625-32.)

### CD3 Cells

The expression “CD3” refers to an antigen which is expressed on T cells as part of the multimolecular T cell receptor (TCR) and is composed of four distinct chains: CD3-epsilon, CD3-delta, CD3-zeta, and CD3-gamma. In mammals, the complex contains a CD3γ chain, a CD3δ chain, and two CD3ε chains. These chains associate with the T-cell receptor (TCR) and the ζ-chain (zeta-chain) to generate an activation signal in T lymphocytes. The TCR, ζ-chain, and CD3 molecules together constitute the TCR complex. Human CD3-epsilon (hCD3ε) comprises the amino acid sequence as set forth in UniProtKB/Swiss-Prot: P07766.2. Human CD3-delta (hCD3δ) comprises the amino acid sequence as set forth in UniProtKB/Swiss-Prot: P04234.1 The CD3 T cell co-receptor helps to activate both the cytotoxic T cells (CD8+ naive T cells) and also T helper cells (CD4+ naive T cells).

The CD3γ, CD3δ, and CD3ε chains are highly related cell-surface proteins of the immunoglobulin superfamily containing a single extracellular immunoglobulin domain. The transmembrane region of the CD3 chains is negatively charged, because it contains aspartate residues, a characteristic that allows these chains to associate with the positively charged TCR chains.

The intracellular tails of the CD3 molecules contain a single conserved motif known as an immunoreceptor tyrosine-based activation motif (ITAM), which is essential for the signaling capacity of the TCR. The intracellular tails of the ζ chain contain 3 ITAM motifs.

### Cancer and NK Cells, Activation of Allogeneic NK Cells

While not wishing to be bound by theory, the multipronged approach and treatment regimen described herein are believed to overcome immunosuppressive effects of T regulatory cell induction, and the combination with activated lymphocytic cells, e.g., activated NK cells, in combination with certain other immune responsive cells, in certain embodiments, is believed to provide enhanced therapeutic efficacy, as described further below. It is noted that the activation and immune training through the activated lymphocytic cells, e.g., activated NK cells, take place in the patient—in vivo, and do not require culturing in vitro.

### Gamma Delta T Cells and Activating Gamma Delta T Cells

Gamma delta T cells (γ∂T cells) represent a small subset of T cells that possess a distinct T cell receptor (TCR) on their surface. A majority of T cells have a TCR composed of two glycoprotein chains called α (alpha) and β (beta)-TCR chains. However, in γδT cells, the TCR is made up of one γ chain and one δ chain. This group of T cells is much less common (5% of total T cells) than the αβT cells, but are found at their highest abundance in the gut mucosa, within a population of lymphocytes known as intraepithelial lymphocytes (IELs). The antigenic molecules that activate γδT cells are still widely unknown. However, γ∂T cells are not MHC restricted and seem to be able to recognize whole proteins rather than requiring peptides to be presented by MHC molecules on antigen presenting cells. Some recognize MHC class IB molecules though. Human V.gamma.9/V.delta.2 T cells, which constitute the major γδT cell population in peripheral blood, are unique in that they specifically and rapidly respond to a small non-peptidic microbial metabolite, HMB-PP, an isopentenyl pyrophosphate precursor.

Methods for activating gamma delta T cells have been described, for example in Deniger et al. Clin Cancer Res. 2014 November 15; 20(22): 5708-5719. None of the methods described herein will utilize feeder cell systems, but will instead rely on cytokine, and/or bead activation of the cells, which provides an advantage in terms of lowered risk of contamination, ease of activation, and increased speed in delivery of the activated cells to the patient.

An example of an activation cocktail for activating the gamma delta T cells includes the following:

500 IU/ml IL-2

20 ng/ml HM-BPP

CD3/CD28 T cell activation beads (1:2 bead to cell ratio)

24 hour incubation.

### Cell Sources

Peripheral blood mononuclear cells (PBMCs) can be isolated by Ficoll-Hypaque density gradient centrifugation of samples obtained from discarded, de-identified leukocyte reduction filters (American Red Cross), or blood donations from healthy volunteers with informed consent. Descriptions of cell populations, sources and methods for selecting or enriching for desired cell types can be found, for example in: U.S. Pat. No. 9,347,044. Populations of cells for use in the methods described herein for treating mammals must be species matched, such as human cells. The cells may be obtained from an animal, e.g., a human patient. If the cells are obtained from an animal, they may have been established in culture first, e.g., by transformation; or more preferably, they may have been subjected to preliminary purification methods. For example, a cell population may be manipulated by positive or negative selection based on expression of cell surface markers; stimulated with one or more antigens in vitro or in vivo; treated with one or more biological modifiers in vitro or in vivo; or a combination of any or all of these. In an illustrative embodiment, a cell population is subjected to negative selection for depletion of non-T cells and/or particular T cell subsets. Negative selection can be performed on the basis of cell surface expression of a variety of molecules, including B cell markers such as CD19, and CD20; monocyte marker CD14; the NK cell marker CD56. Alternately, a cell population may be subjected to negative selection for depletion of non-CD34.sup.+ hematopoietic cells and/or particular hematopoietic cell subsets. Negative selection can be performed on the basis of cell surface expression of a variety of molecules, such as a cocktail of antibodies (e.g., CD2, CD3, CD11b, CD14, CD15, CD16, CD19, CD56, CD123, and CD235a) which may be used for separation of other cell types, e.g., via MACS or column separation.

Populations of cells include peripheral blood mononuclear cells (PBMC), whole blood or fractions thereof containing mixed populations, spleen cells, bone marrow cells, tumor infiltrating lymphocytes, cells obtained by leukapheresis, biopsy tissue, lymph nodes, e.g., lymph nodes draining from a tumor. Suitable donors include immunized donors, non-immunized (naive) donors, treated or untreated donors. A “treated” donor is one that has been exposed to one or more biological modifiers. An “untreated” donor has not been exposed to one or more biological modifiers.

Methods of obtaining populations of cells comprising a T cell are well known in the art. For example, peripheral blood mononuclear cells (PBMC) can be obtained as described according to methods known in the art. Examples of such methods are set forth in the Examples and is discussed by Kim et al. (1992); Biswas et al. (1990); Biswas et al. (1991).

It is also possible to obtain a cell sample from a subject, and then to enrich it for a desired cell type. For example, PBMCs can be isolated from blood as described herein. Counter-flow centrifugation (elutriation) can be used to enrich for T cells from PBMCs. Cells can also be isolated from other cells using a variety of techniques, such as isolation and/or activation with an antibody binding to an epitope on the cell surface of the desired cell type, for example, some T-cell isolation kits use antibody conjugated beads to both activate the cells and then allow column separation with the same beads. Another method that can be used includes negative selection using antibodies to cell surface markers to selectively enrich for a specific cell type without activating the cell by receptor engagement.

Bone marrow cells may be obtained from iliac crest, femora, tibiae, spine, rib or other medullary spaces. Bone marrow may be taken out of the patient and isolated through various separations and washing procedures. A known procedure for isolation of bone marrow cells comprises the following steps: a) centrifugal separation of bone marrow suspension in three fractions and collecting the intermediate fraction, or buffycoat; b) the buffycoat fraction from step (a) is centrifuged one more time in a separation fluid, commonly Ficoll (a trademark of Pharmacia Fine Chemicals AB), and an intermediate fraction which contains the bone marrow cells is collected; and c) washing of the collected fraction from step (b) for recovery of re-transfusable bone marrow cells.

If one desires to use a population of cells enriched in T cells, such populations of cells can be obtained from a mixed population of cells by leukapheresis and mechanical apheresis using a continuous flow cell separator. For example, T cells can be isolated from the buffy coat by any known method, including separation over Ficoll-Hypaque™ gradient, separation over a Percoll gradient, or elutriation.

Phenotypic Analysis of NK Cells. PBMCs can be stained for flow cytometric analysis using fluorochrome-conjugated antibodies as previously described by Kim et al. (Proc Natl Acad Sci USA 105(8):3053-3058 (2008)). Antibodies to detect the following proteins from Beckman Coulter can be utilized: (CD56 (N901), NKG2A (Z199), NKp44 (Z231)), BD Biosciences (CD3 zeta (UCHT1), CD16 (3G8), KIR2DL2/3 (CH-L), KIR3DL1 (DX9), NKp46 (9E2), NKp30 (p30-15), CD11a (G43-25B), CD11b (ICRF44), CD11c (B-1y6), NKG2D (1D11), CD161(DX12), DNAM-1 (DX11), CD57 (NK-1), CD25 (M-A251), IFN-.gamma. (B27), TNF-.alpha. (Mab11), CD107a (H4A3), Granzyme A (CB9), Granzyme B (GB11)), Biolegend (CD14 (HCD14), CD19 (HIB19), CD2 (RPA-2.10), 2B4 (C1.7), NTB-A (NT-7), CRACC (162.1), CD69 (FN50)), eBiosciences (KIR2DL1 (HP-MA4), Perforin (dG9)), and R&D (NKG2C (134591)).

In some embodiments, the following embodiments are provided:
1. A method of treating a patient with HIV, cancer, a viral infection, or a bacterial infection, the method comprising administering an effective amount of a lymphocytic cellular composition comprising activated natural killer (NK) cells to the patient.
2. A method for increasing an immune response in a patient in need thereof, comprising administering an effective amount of a lymphocytic cellular composition comprising activated NK cells to the patient.
3. A method of inhibiting HIV replication in a patient, the method comprising administering to the subject with HIV an effective amount of a lymphocytic cellular composition comprising activated natural killer (NK) cells to the patient.
4. A method of inhibiting tumor growth in a patient, the method comprising administering to the patient with the tumor an effective amount of a lymphocytic cellular composition comprising activated natural killer (NK) cells to the patient.
5. A method of inhibiting a viral or bacterial replication or reproduction in a subject having a viral or bacterial infection, the method comprising administering to the subject with the viral or bacterial infection an effective amount of a lymphocytic cellular composition comprising activated natural killer (NK) cells to the patient.
6. The method of any one of embodiments 1-5, wherein the cellular composition further comprises activated gamma delta T cells (GDT cells).
7. The method of any one of embodiments 1-5, wherein the cellular composition further comprises invariant natural killer T cells (iNKT cells).
8. The method of any one of embodiments 1-5, wherein the cellular composition further comprises CD3+ T cells.
9. The method of any of embodiments 1-8, wherein prior to administration to the patient, the lymphocytic cellular composition is activated by mixing/incubating/contacting the cells with at least one cytokine and optionally soluble fibroblast growth factor receptor 1 (sFGFR1).
10. The method of embodiment 9, wherein the cytokine is selected from the group consisting of IL-2, IL-15, IL-21, Flt3-L, stem cell factor (SCF), IL-7, IL-12, and IL18, and any combination thereof.
11. The method of embodiment 10, wherein the cytokine is incubated/mixed/contacted with the cellular composition for between about 6-24 hours.
12. The method of embodiment 10, wherein the amount of cytokine incubated with the cellular composition is between about 100-1000 IU/ml.
13. The method of any of embodiments 1-12, wherein the method further comprises pre-conditioning the patient prior to administering the lymphocytic cellular composition by administering at least one lympho-suppressive agent, chemotherapeutic agent, or immunosuppressive agent for between 3-5 days prior to administering the lymphocytic cellular composition.
14. The method of embodiment 13, wherein the lympho-suppressive or chemotherapeutic agent comprises any one of 6TG, 6-MMP, one or more purine analogues selected from the group consisting of clofarabine, fludarabine, and cytarabine, or any combination thereof.
15. The method of embodiment 14, wherein the dose of the lympho-suppressive or chemotherapeutic agent ranges from about 5 mg/m² to about 50 mg/m².
16. The method of embodiment 13, wherein the chemotherapeutic or immunosuppressive agent comprises any cyclophosphamide, rituximad, a steroid or any combination thereof.
17. The method of embodiment 16, wherein the steroid is a glucocorticoid steroid.
18. The method of embodiment 17, wherein the steroid is prednisone.
19. The method of embodiment 16, wherein the dose of the chemotherapeutic agent or immunosuppressive agent ranges from about 20 mg/m2 to about 1000 mg/m2.
20. The method of embodiment 13, wherein the method further comprises administering to the patient interferon-alpha (IFN-α), or a biological equivalent thereof, at a dosage of from about 1×10⁶ IU/m²/d to about 10×10⁶ IU/m²/d, two days and/or 1 day prior to administering the lymphocytic cellular composition.
21. The method of any of embodiments 1-20, wherein the method further comprises administering to the patient IL-2 on the day of administration of the lymphocytic cellular composition and on the first day following administration of the lymphocytic cellular composition, and optionally continuing for between 3-14 additional days following administration of the lymphocytic cellular composition, in a dosage of about 3-6×10⁶ IU/m² per dose.
22. The method of any of embodiments 1-21, wherein the method further comprises administering to the patient a COX-2 inhibitor or a nonsteroidal anti-inflammatory drug (NSAID), or a combination thereof, on the day of administration of the lymphocytic cellular composition, and continuing for between at least 14 and 60 days following administration of the lymphocytic cellular composition.
23. The method of embodiment 22, wherein the COX-2 inhibitor is celecoxib or rofecoxib, and the nonsteroidal anti-inflammatory drug is selected from the group consisting of aspirin, indomethacin, ibuprofen, naproxen, piroxicam, and nabumetone.
24. The method of any of embodiments 1-23, wherein the activated NK cells are allogeneic to the patient.
25. The method of any of embodiments 1-24, wherein the NK cells are not HLA-matched with the patient.
26. The method of any of embodiments 1-25, wherein the dosage of activated cells ranges from about 3-50×10⁶.
27. The method of any of embodiments 1-26, wherein the viral infection is selected from the group consisting of viral infections caused by retroviruses (e.g., human T-cell lymphotrophic virus (HTLV) types I and II and human immunodeficiency virus (HIV)), herpes viruses (e.g., herpes simplex virus (HSV) types I and II, Epstein-Ban virus and cytomegalovirus), arenaviruses (e.g., lassa fever virus), paramyxoviruses (e.g., morbillivirus virus, human respiratory syncytial virus, and pneumovirus), adenoviruses, bunyaviruses (e.g., hantavirus), coronaviruses, filoviruses (e.g., Ebola virus), flaviviruses (e.g., hepatitis C virus (HCV), yellow fever virus, and Japanese encephalitis virus), hepadnaviruses (e.g., hepatitis B viruses (HBV)), orthomyoviruses (e.g., Sendai virus and influenza viruses A, B and C), papovaviruses (e.g., papillomaviruses), picornaviruses (e.g., rhinoviruses, enteroviruses and hepatitis A viruses), poxviruses, reoviruses (e.g., rotaviruses), togaviruses (e.g., rubella virus), and rhabdoviruses (e.g., rabies virus), and any combination thereof.
28. The method of any of embodiments 1-26, wherein the cancer is a hematological or hematogenous cancer selected from the group consisting of acute leukemia, acute myelocytic leukemia, acute myelogenous leukemia, myeloblastic leukemia, promyelocytic leukemia, myelomonocytic leukemia, monocytic leukemia, erythro leukemia, chronic leukemia, chronic myelocytic (or granulocytic) leukemia, chronic myelogenous leukemia, chronic lymphocytic leukemia, polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia, and any combination thereof.
29. The method of any of embodiments 1-26, wherein the cancer is a solid tumor selected from the group consisting of fibrosarcoma, myxosarcoma, liposarcoma, chondrosarcoma, osteosarcoma, and other sarcomas, synovioma, mesothelioma, Ewing's tumor, leiomyo sarcoma, rhabdomyo sarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases.
30. The method of any of embodiments 1-26, wherein the bacterial infection is caused by one of the pathogenic bacterial species Bacteroides, Clostridium, Streptococcus, Staphylococcus, Pseudomonas, Haemophilus, Legionella, Mycobacterium, Escherichia, Salmonella, Shigella, or Listeria.
31. The method of any of embodiments 1-26, wherein graft versus host disease (GVHD) is decreased or eliminated, while graft versus tumor (GVT) or graft versus virus (GVV) is increased in the patient.
32. The method of embodiments 1-20, wherein administration of IL-2 is counter-indicated and not administered following administration of the lymphocytic cellular composition comprising activated NK cells, when the patient has lymphocytic or lymphoblastic leukemia or lymphoma.
33. The method of any of embodiments 1-26, wherein at a time-frame of from 4-14 days following administration of the lymphocytic cellular composition comprising activated NK cells, the patient is further administered an immune check-point inhibitor, including any one or combination of two check point inhibitors, including an inhibitor of PD-1 or PD-L1 (B7-H1), such as an anti-PD-1 antibody, including nivolumab (Nivolumab from Bristol-Myers Squibb), pembrolizumab/lambrolizumab, also known as MK-3475 (Keytruda from Merck), pidilizumab (Curetech), AMP-224 (Amplimmune), or an anti-PD-L1 antibody, including MPDL3280A (Roche), MDX-1105 (Bristol Myer Squibb), MEDI-4736 (AstraZeneca) and MSB-0010718 C (Merck), an antagonist of CTLA-4, such as an anti-CTLA-4 antibody including anti-CTLA4 antibody Yervoy™ (ipilimumab, Bristol-Myers Squibb), tremelimumab (Pfizer), Ticilimumab (AstraZeneca) or AMGP-224 (Glaxo Smith Kline), or a tumor specific antibody trastuzumab (Herceptin) for breast cancer, rituximab (Rituxan) for lymphoma, or cetuximab (Erbitux).
34. The method of any of embodiments 1-22, wherein the treatment or increasing the immune response is repeated periodically for time frames of from once every month, to once every two months, to once every 3 months, to once every 4 months, to once every 5 months, to once every 6 months, or once every 7 months, or once every 8 months, or once every 9 months, or once every 10 months, or once every 11 months, or once annually as a maintenance treatment for as long as the patient exhibits improvement, or stable/non-progressing disease.
35. The method of any of embodiments 1-26, wherein the method further comprises administering to the patient one or two antihistamine drugs, on the same day, but at least about 2-6 hours prior to administration of the lymphocytic cellular composition comprising activated NK cells to the patient.
36. A method of activating NK cells, the method comprising contacting a cellular composition comprising the NK cells in vitro with at least one cytokine and optionally soluble fibroblast growth factor receptor 1 (sFGFR1).
37. The method of embodiment 36, wherein the cytokine is selected from the group consisting of IL-2, IL-15, IL-21, Flt3-L, stem cell factor (SCF), IL-7, IL-12, and IL18, and any combination thereof.
38. The method of embodiment 36, wherein the cytokine is contacted with the cellular composition for from about 6 to about 24 hours.
39. The method of embodiment 36, wherein the amount of cytokine contacted with the cellular composition is from about 100 IU/ml to about 1000 IU/ml.
40. The method of any one of embodiments 36-39, wherein the composition further comprises gamma delta T cells (GDT cells).
41. The method of any one of embodiments 36-40, wherein the cellular composition further comprises invariant natural killer T cells (iNKT cells).
42. The method of any one of embodiments 36-41, wherein the cellular composition further comprises CD3+ T cells.
43. The method of any one of embodiments 36-42, wherein the NK cells, gamma delta T cells (GDT cells), invariant natural killer T cells (iNKT cells), and/or CD3⁺ T cells are isolated from peripheral blood.
44. A cytokine and optionally soluble fibroblast growth factor receptor 1 (sFGFR1) activated lymphocytic cellular composition comprising activated natural killer (NK) cells.
45. The composition of embodiment 44, wherein the cytokine activated composition a IL-2, IL-15, IL-21, Flt3-L, stem cell factor (SCF), IL-7, IL-12, and IL18, and any combination thereof, activated composition.
46. The composition of embodiments 44 and 45, wherein the composition further comprises gamma delta T cells (GDT cells), invariant natural killer T cells (iNKT cells), and/or CD3⁺ T cells.
47. The composition of any one of embodiments 44-46, wherein the NK cells are peripheral blood NK cells.
48. The composition of any one of embodiments 44-47, wherein the gamma delta T cells (GDT cells), invariant natural killer T cells (iNKT cells), CD3⁺ T cells are peripheral blood gamma delta T cells (GDT cells), invariant natural killer T cells (iNKT cells), and CD3⁺ T cells.
49. A cytokine and optionally soluble fibroblast growth factor receptor 1 (sFGFR1) activated lymphocytic cellular composition comprising activated natural killer (NK) cells for treating a patient with HIV, cancer, a viral infection, or a bacterial infection.
50. A cytokine and optionally soluble fibroblast growth factor receptor 1 (sFGFR1) activated lymphocytic cellular composition comprising activated natural killer (NK) cells for the preparation of a medicament for treating a patient with HIV, cancer, a viral infection, or a bacterial infection.

The following examples are illustrative, but not limiting, of the compounds, compositions and methods described herein. Other suitable modifications and adaptations known to those skilled in the art are within the scope of the embodiments provided herein.

### EXAMPLE 1

Options for Treating Cancer Patients, as well as Viral Infections including HIV

As an initial step, NK cells are obtained or purified from any suitable commercial source—and the cells are allogeneic, as described herein and in the above sections.

As described herein, there are four different infusion options for administering therapeutic cellular compositions to the patient. These therapeutic cellular compositions include the following cellular components:

activated NK cells; or

activated NK cells+activated GDT cells; or

activated NK cells+activated GDT cells+iNKT cells; or

activated NK cells+CD3 T cells.

### Stage 1: Patient Pre-Conditioning Options

Depending on the condition of the patient, and the stage of disease, as described in FIG. 1, the patient is pre-treated using one of the following pre-conditioning protocols listed in Tables 1-5. The day 0 reference is the infusion day (e.g. infusion of the activated lymphocytic cell composition).

TABLE 1

| | |
|---|---|
| Day −7 to day −3 | Fludarabine 25 mg/m²/day IV |
| Day −4 | Cyclophosphamide 1000 mg/m² IV |

TABLE 2

| | |
|---|---|
| Day −5 to day −1 | Fludarabine 15 mg/m²/day IV |
| Day −2 | Cyclophosphamide 1000 mg/m² IV |

TABLE 3

| | |
|---|---|
| Day −3 and day −2 | Cyclophosphamide 1000 mg/m² IV |

TABLE 4

| | |
|---|---|
| Day −5 to day −2 | Fludarabine 25 mg/m²/day IV |

TABLE 5

| | |
|---|---|
| Day −5 to day −1 | Cyclophosphamide 200 mg/d PO |

In certain embodiments, following the pre-treatment regimen of any of Tables 1-5, the patient is then treated at Day −2 and day −1 with interferon alpha (IFN-α) 3×10⁶ IU/m²/d by subcutaneous (SC) injection. The amount of IFN-α can range from about 1×10⁶ to about 5×10⁶ IU/m²/d by SC injection.

In some embodiments, the method further comprises administering to the patient one or two antihistamine drugs (in a standard dose, such as 25 mg promethazine and 25 mg diphenhydramine), on the same day, but at least about 2-6 hours prior to administration of the lymphocytic cellular composition comprising activated NK cells to the patient.

### In Vitro Activation of the Lymphocytic Therapeutic Cells

In certain embodiments, the activation cocktail can include IL-2, IL-12, IL-15, IL-18 and IL-21 singly, or in any combination. The activation process is usually overnight, minimum 12 hours, typically not longer than 24 hours.

In certain embodiments, soluble FGF1 receptor is used as an activation agent (FGFR1, specifically the extracellular domain of FGF1 receptor, purified from the construct as shown in FIG. 4) in an exemplary concentration of 33.33 nano-molar per liter (or 33.33 pico-molar per ml). The exposure of the lymphocytic cells to the extracellular domain of FGF1 receptor functions to activate the CD56+ NK cell population and drives them to become a nearly pure cytotoxic NK cell composition (e.g. at least 80% cytotoxic NK cells) (as opposed to a mixed population of cytotoxic and regulatory NK cells). The activation cocktail can simply be added into the buffer (or media) the cells are kept in after being isolated. In certain embodiments, freshly isolated cells from fresh leukapheresis material are utilized for the source of NK cells. However, in certain embodiments, other cell collection/isolation/and/or preservation scenarios can be utilized as the cell source. For example, the cells can be cryo-preserved before or —more preferably—after the activation step. Activation using the extracellular domain of FGF1 receptor can be used for activating any of the lymphocytic cells described herein, including the NK cells, GDT cells, and iNK cells.

### Exemplary Method Utilizing Fresh Cellular Material:

Freshly mobilized leukapheresis material is processed for the isolation of the “target cell population” (any of the 4 options described above).
The isolated cells are suspended in a buffer solution or media with the activation cocktail. PBS with 5-20% HSA (Human Serum Albumin) is the typical buffer solution, but other similar options can be utilized as well.
The cells are washed after 12+ hours and suspended again in an injectable solution. Typically, this buffer solution is PBS with 5-20% HSA, but can be any suitable solution. In certain embodiments, the patient's own serum or plasma can be utilized as the infusion media.
The cells are infused into the patient after the pre-conditioning process described in Tables 1-5 along with the optional day −2 and day −1 interferon alpha treatment.

It is noted that once the lymphocytes (NK or other) are in vitro activated with IL-2 or any other combination cocktail that includes IL-2, their cytotoxic potency becomes dependent on IL-2. This means that without a certain level of IL-2 in the patient's blood, the activated lymphocytes (NK or other) start losing their potency. Thus, in the cases where IL-2 is utilized in the in vitro activation cocktail, the patient should be given IL-2 injections on the day of the cell infusion and continuing for at least 4 days (up to 14 days) following the cell infusion. The typical IL-2 dose is from about 3-6 million IU/m² per day as subcutaneous injections.

Additionally, IL-2 activation is not utilized to activate the NK cells or other therapeutic compositions in patients with lymphocytic or lymphoblastic leukemia or lymphoma (See FIG. 1, options for lymphoblastic leukemia or lymphoma patients). This different course of not using IL-2 for activation for the therapeutic cell compositions in these lymphocytic or lymphoblastic leukemia or lymphoma patients avoids putting these cancers into overdrive, and comprising the potency of the therapeutic cell composition. Thus, as shown in FIG. 1, no IL-2 activation would be utilized for these patients.

Stage 2: Options for Lymphocytic Cell Populations for Infusion into a Patient, Depending upon Disease Stage or Condition.

As shown in FIG. 1, there are four different cell populations, or “cell cocktails” that depending on the disease and stage are utilized in the infusion, as an “activated lymphocytic composition.” The various make-ups of these compositions and variations, are described below.

Additionally, certain components or embodiments of these activated lymphocytic compositions can be provided in a kit. For example, any of the four lymphocytic compositions could be provided as a frozen composition and packaged as a kit, alone or along with separate containers of any of the other agents from the pre-conditioning or post-conditioning steps, and optional instructions for use.

Some embodiments are also directed to any of the aforementioned cellular compositions in a kit. In some embodiments, the kit may comprise ampoules, disposable syringes, capsules, vials, tubes, or the like. In some embodiments, the kit may comprise a single dose container or multiple dose containers comprising the topical formulation of embodiments herein. In some embodiments, each dose container may contain one or more unit doses. In some embodiments, the kit may include an applicator. In some embodiments, the kits include all components needed for the 3 stages of conditioning/treatment. In some embodiments, the cellular compositions may have preservatives or be preservative-free (for example, in a single-use container).

### Lymphocytic Cell Infusion

For the lymphocytic cell infusion, for example with NK cells, at Day 0, the patient is administered the in vitro activated allo-NK infusion (1-50×10⁶ cells/kg). The cell dose can vary broadly from about 1-50 million cells per kg. As described below, the lymphocytic cell infusion mixture may comprise any of the following combinations:

activated NK cells; or

activated NK cells+activated GDT cells; or

activated NK cells+activated GDT cells+iNKT cells; or

activated NK cells+CD3 T cells.

1. A Cellular Population of “Substantially” Pure NK Cells

In certain embodiments, a substantially pure NK cell population (“donor allogeneic NK cells” ranging from at least about 75% NK cells, to around about 98% NK cells) is used for the treatment of minimal residual disease in certain cancer patients. The isolation of essentially pure NK cells can be done manually or using cliniMACS, via negative or positive selection of CD16⁺CD56⁺ NK cells. Because NK cells generally cannot proliferate (or they don't proliferate as much as other lymphocytes) their effect is limited and their lifespan is also very limited. These activated NK allogeneic cells survive in the host/recipient body anywhere between about 5-15 days. That provides the ability to mitigate the levels of adverse reaction associated with tumor death, like tumor lysis syndrome, swelling/inflammation of the tumors, etc. Importantly, the complication of graft-versus host disease (GVHD) is eliminated because of the antigen independence of NK cells combined with their inability to grow and their short lifespan.

The isolation of essentially pure NK cells can be done manually or using cliniMACS, via negative or positive selection of CD16⁺CD56⁺ NK cells, and as described above and herein (See also methods in Slavin, S. et al. Cancer Immunol. Immunther. (2010) 59:1511-1519).

2. A Lymphocytic Cellular Composition Comprising a Mixture of Pure NK and GDT Cells (Preferably, both cell types are activated, either together or separately).

Since the donor gamma delta T cells (γδ T cells, or GDT cells) function essentially like donor NK cells with regard to being MHC independent and killing through stress signals of the target cells, they can also be used in combination with essentially pure donor NK cells for treating certain patients. A difference between the donor NK and donor GDT cells is that donor GDT cells expand in vivo.). The infused NK Cells cannot proliferate. Since GDT cells can behave like T cells, and because they can expand, and they have a much longer lifespan in the patient than the donor NK cells. Using donor GDT cells is beneficial in the cases where a transient donor micro-chimerism is desired, which enables a much more potent and longer lasting graft-versus-tumor (GVT) (or graft-versus-leukemia or graft-versus-virus) effect. The cell “product” mixture can be prepared by isolating NK and GDT cells together from a leukapheresis material. Alternatively, they can be isolated separately and activated separately, and then mixed together in a desired ratio for the purposes of controlling the dose of each population on a case by case basis.

3. NK+GDT+iNKT Cellular Composition Cocktail (preferably, both the NK and GDT cell types are activated, either together or separately).

The composition comprising NK+GDT+iNKT cells is similar to the composition comprising NK and GDT cells because the iNKT population is a very small percentage in PBMCs. However, the isolation process is slightly different. Thus, in certain embodiments, the process to isolate this mixture of NK+GDT+iNKT cells is through depletion of neutrophils, monocytes, AB T cells and B cells, and collecting the leftover components. For example, the leukapheresis material is subjected to a combined CD14, CD15, CD19, CD4 and CD8 depletion. The leftover cells are a mixture of NK+GDT+iNKT cells. This is isolation process is very practical but it doesn't allow for control of the dose of each population of cell types individually.

It is noted that iNKT cells are CD1d-restricted lipid-sensing innate T cells that express the transcription factor PLZF. (Nat Immunol. 2015 January; 16(1):85-95). iNKT cells have a semi-invariant αβTCR and recognize CD1d-presented lipid antigens. Unlike adaptive MHC-restricted T cells, they display an effector and memory phenotype at steady-state, which renders them poised for immediate effector function. Because of their rapid response and basal expression of NK receptors they are considered “innate” T cells. iNKT cells characteristically express high levels of the BTB-POZ transcription factor PLZF, encoded by Zbtb16, this transcription factor was proposed to define the iNKT cell lineage. PLZF is also expressed by human MAIT cells, another population of semi-invariant T cells, as well as an innate subset of γδ T cells. Thus, PLZF expression is associated with T cells with limited TCR diversity and is thought to be responsible for the innate phenotype and rapid cytokine response of these cells. (See, Anticancer Res. 2018 July ;38(7):4233-4239).

4. NK+CD3 Cellular Composition Cocktail (activated NK cells in combination with CD3 cells)

Previous treatments using allogeneic lymphocytes in allo-transplants caused GVHD in these patients. Thus, practitioners have been wary of using CD3 mixtures. In this treatment regimen, the potential for GVHD is mitigated by the pre-conditioning steps that effectively suppress the patient's immune system, temporarily (e.g. lympho-suppression of about 5-7 days). While not wishing to be bound by theory, it is believed that as long as the number of CD3 cells that are infused is not high enough to overcome the host immune system at the end of the 5-7 day lympho-suppression period that is the result of the pre-conditioning step, there is no danger of GVHD in the patient. Another reason that there is no danger of GVHD is because GVT (graft versus tumor) (or GVL or GVV (graft versus virus)) always comes before GVHD. That means, as long as the patient has some tumor burden with tumor antigens that serve as a target for the CD3 allogeneic cells, there will be “no time for GVHD” to develop because the allogeneic cells will be busy attacking the invader cells, e.g. the cancer/virus/bacterial cells. There is a very small risk of GVHD if there is a possibility of completely wiping out the target cancer or virally or bacterially infected cells—within one single treatment. For example, if the patient is in minimal residual disease and is treated with a combination of NK+CD3 cells, and the allogeneic cells kill all the cancer cells within the first few days before the patient's immune system rebounds from the effects of pre-conditioning. It is believed that since CD3 cells live longer and they expand, and they attack anything foreign, it is expected that when there are no cancer cells to attack, they will start attacking the healthy host cells and they will keep proliferating. This could cause an acute and probably low grade GVHD. However, the situation to be avoided is one where there is an initial infusion of a higher dose of CD3 cells, and by the time the pre-conditioning wears off they have expanded to a degree that the host immune system can't reject them anymore, then a more serious GHVD could result. Thus, this combination of activated NK cell and CD3 cell treatment is only used in patients with very heavy tumor burden like metastatic solid tumor patients and advanced or metastatic hematological cancer patients (See FIG. 1). The addition of CD3 cells into the treatment actually creates a longer lasting donor micro-chimerism and a better tumor-infiltration of allogeneic cells. The alloreactivity of the allogeneic cells indirectly helps the host immune system to infiltrate into the tumor microenvironment easier. This procedure induces a much stronger host anti-tumor immunogenicity.

Stage 3: Post-Infusion Options. As Shown in FIG. 1, Three Options are Available for Post-Infusion Treatment (or Post-infusion Conditioning). The post-infusion regimens function to prevent or diminish the patient/s immune exhaustion and/or immune tolerance.

1. Post-Infusion In Vivo IL-2 Activation.

In certain patients, there will be in vivo IL-2 activation achieved by administering from day 0 (the cell infusion day) to about day 5 post-infusion, IL-2 to the patient following the infusion of activated NK cells. Post-infusion, in vivo IL-2 administration, is only indicated when the NK cells are in vitro activated with a cocktail that includes IL-2. The amount of IL-2 can vary, but is typically about 6×10⁶ IU/m²/day as SC injections. The amount of IL-2 administered to the patient can vary from between about 1×10⁶ IU/m²/day to about 6×10⁶ IU/m²/day and is typically administered subcutaneously (SC). This post-infusion step is not administered in cancers where injecting the patient with IL-2 is not recommended, such as lymphocytic or lymphoblastic leukemia or lymphoma patients. For these patients, no IL-2 will be administered in vivo post-infusion. It is expected that longest time a patient would be administered IL-2 in vivo post-infusion, would be 7 days.

2. Post-Infusion Administration of COX-2 Inhibitors

While not wishing to be bound by theory, post-infusion treatment options relating to administering COX-2 inhibitors (specific or non-specific) to the patient, serve to block PGE2 and FOXP3 pathways and thus hinder the Treg differentiation and production to avoid immune exhaustion and immune tolerance, two complicating factors associated with many cell-based therapies, especially ones involving IL-2 activation. Additionally, the post-infusion treatments are helpful in managing the infusion-related fever, and pain associated with the activated lymphocytic cell infusion. These specific or non-specific COX-2 inhibitor agents can be administered orally, in a post-infusion or post-conditioning regimen ranging in time period ranging from day 0 (the cell infusion day) to any period ranging from about 3-4 weeks, in an amount of about 200-400 mg/day for the 3-4 week time-frame.

3. Post-Infusion Administration of PGE2 Inhibitors

Similarly, other PGE2 inhibitors, which include non-specific COX-2 inhibitors such as aspirin, indomethacin (Indocin), ibuprofen (Advil, Motrin), naproxen (Naprosyn), piroxicam (Feldene), and nabumetone (Relafen) can also be administered to the patient post-activated lymphocytic cell infusion, in accordance with FIG. 1. These compounds have a similar effect to the COX-2 inhibitors. These agents can be administered orally, in a post-infusion or post-conditioning regimen for a time period ranging from day 0 (the cell infusion day) to any period ranging from about 3-4 weeks, in an amount of about 80-300 mg/day.

Additionally, in certain patients, it is expected that this treatment regimen would be repeated periodically to boost the immune system response to the tumors or infectious agent/s. Such periodic treatment can vary from once every month, to once every two months, to once every 3 months, to once every 4 months, to once every 5 months, to once every 6 months, or once every 7 months, or once every 8 months, or once every 9 months, or once every 10 months, or every 11 months, or once annually as a maintenance treatment for as long as the patient requires.

### EXAMPLE 2

### Immune Check Point Inhibitors Administered Following Activated Lymphocyte Treatment

In certain embodiments, the patient is treated with one of the activated lymphocyte cell regimens described herein, and then further administered an immune check-point inhibitor at a time-frame of from 4-14 days following the activated lymphocyte cell composition. While not wishing to be bound by theory, it is believed that since the donor activated lymphocytic cells have a life span of around 4/5-15 days in the recipient patient, as they begin dying they function to present tumor antigens and act like a vaccine in the patient. At this time, (about 4-14 days following activated lymphocytic cell administration), the patient can be administered any one or combination of two of the immune cell checkpoint inhibitors, e.g., molecules that send an inhibitory signal to the immune system. Following the administration of the immune cell checkpoint inhibitor, the post-conditioning treatment options described herein would be followed. The dosing of the immune cell checkpoint inhibitor would be the standard dosing. Additionally, in certain patients, it is expected that this treatment would be repeated periodically to boost the immune system response to the tumors. Such periodic treatment can vary from once every month, to once every two months, to once every 3 months, to once every 4 months, to once every 5 months, to once every 6 months, or once every 7 months, or once every 8 months, or once every 9 months, or once every 10 months, or every 11 months, or once annually as a maintenance treatment for as long as the patient requires.

Examples of such agents include inhibitors of PD-1 or PD-L1 (B7-H1), such as anti-PD-1 antibodies, including nivolumab (Nivolumab from Bristol-Myers Squibb) and pembrolizumab/lambrolizumab, also known as MK-3475 (Keytruda from Merck), pidilizumab (Curetech), AMP-224 (Amplimmune), and anti-PD-L1 antibodies, including MPDL3280A (Roche), MDX-1105 (Bristol Myer Squibb), MEDI-4736 (AstraZeneca) and MSB-0010718 C (Merck). Other checkpoint inhibitors include antagonists of CTLA-4, such as anti-CTLA-4 antibodies. An exemplary anti-CTLA4 antibody is Yervoy™ (ipilimumab) marketed by Bristol-Myers Squibb. Other exemplary CTLA-4 antibodies include tremelimumab (Pfizer), Ticilimumab (AstraZeneca) and AMGP-224 (Glaxo Smith Kline).

Metastatic Breast Cancer Patient treated with activated NK cells followed by Nivolumab

A patient with metastatic breast cancer was treated with the activated lymphocyte cell regimen described herein, and then further administered an immune check-point inhibitor at a time-frame of from about 8 days following administration of the activated lymphocytic cell composition (the timing for administration of the check-point inhibitor can be anywhere from about 4-15 days following administration of the activated lymphocyte cell composition). The treatment was well-tolerated with only grade 1 side effects experienced. Additionally, the initial results show that all metastases shrunk to nearly undetectable levels. A summary of this data, as well as additional results and regimens are shown in FIGS. 3A-E.

### EXAMPLE 3 HIV Treatment Options

Individuals with HIV that take their medicine-antiretroviral therapy (ART) as prescribed and maintain an undetectable viral load can live long, healthy lives and have effectively no risk of sexually transmitting HIV to a partner. However, a significant subset of HIV patients has evidence of drug resistance due to suboptimal therapy prior to combination treatment and/or poor adherence. This is a particular problem in major cities in the United States in California, New York, Illinois, Florida and certain countries, for example Brazil and Thailand, where there was widespread use of mono- and duo-therapy in the 1980s and 1990s^{]}. The substantial number of HIV patients fail to adhere due to a variety of reasons, including adverse side effects, toxicity, drug resistance, drug abuse, mental disorders, socioeconomic status, literacy, and social stigma^{[4]}. Such patients have ongoing viral replication, often with significant levels of plasma viremia, and, therefore, have severely compromised immune systems with limited options and an inexorable pathway to significant morbidity and, ultimately, death.

Despite significant efforts, little progress has been made to ameliorate patients with unsuppressed plasma viremia.

At present, pre-clinical and clinical models of HIV disease progression suggest that HIV-associated disruption of the gastrointestinal tract results in microbial translocation across a compromised intestinal barrier and subsequent chronic immune activation, disease progression, and increased mortality in HIV disease. Gamma delta (γδ) T cells are an ‘innate’ T cell type that expresses a semi-invariant T cell receptor (TCR). The differential usage of the Vδ1 or Vδ2 genes in the rearranged TCR differentiate two main subsets of human γδ T cells^{[7]}.The recognition of both microbial products and stressed host cells allows γδ T cells to play an important role in immune responses against infections in general and viruses in particular^{[8-10]}. While Vδ2+ cells primarily circulate in blood, Vδ1+ cells primarily localize within the mucosa of the gut as intraepithelial lymphocytes (IELs) and help to maintain epithelial function^{[9]}. γδ T cells possess a combination of both innate and adaptive immune cell qualities rendering them attractive for potential immunotherapy approaches^{[11-13]}. They can produce inflammatory cytokines, directly lyse infected or malignant cells, and establish a memory response to attack pathogens upon re-exposure. γδ T cells are defined by expression of γ and δ heterodimer of T cell receptor (TCR) chains (TCRγ/TCRδ) that directs intracellular signaling through associated CD3/TCR complexes^{[14]}. The γδ T-cell lineage (1-5% of circulating T cells) can be contrasted to the more prevalent αβ T cell lineage (˜90%) in peripheral blood, which expresses TCRα/TCRβ heterodimers and also signals through associated CD3/TCR complexes^{[15,16]}. CD4 and CD8 co-receptors on αβ T cells assist binding of TCRαβ chains to the major histocompatibility complex (MHC) presenting various peptides be presented on the formed CD3/TCR complexes^{[17-19]}. In contrast, TCRγδ directly binds to an antigen's superstructure independent of the MHC/peptide complexes and, as a result, CD4 and CD8 are uncommon on γδ T cells^{[20,21]}. Given that antigen recognition is achieved outside of MHC/peptide-restriction, γδ T cells have predictable immune effector functions mediated through their TCR and have potential use as universal allogeneic adoptive cell therapies^{[22]}.

It has been demonstrated that γδ T cells do not cause significant alloreactivity and, therefore, do not induce severe graft versus host disease.

Natural Killer (NK) cells are innate lymphoid cells that provide an extended surveillance against tumor-transformed or viral-infected cells in the absence of antigen recognition. They are important effectors of innate immunity playing a key role in the eradication and clearance of viral infections. Moreover, NK cells are considered to be the bridge between innate and adaptive immune system cells. Over the recent years, several studies have shown that HIV-1 pathologically changes NK cell homeostasis and hampers their antiviral effector functions. Moreover, high levels of chronic HIV-1 viremia markedly impair those NK cell regulatory features that normally regulate the cross-talks between innate and adaptive immune responses. The presence of specific haplotypes for NK cell receptors as well as the engagement of NK cell antibody dependent cell cytotoxicity (ADCC) have been reported to control HIV-1 infection.

The genetically encoded inhibitory receptors on NK cells, including the killer immunoglobulin-like receptors (KIRs) heavily influence NK cell activation, which is governed by the balance between activating and inhibitory signals. Interactions between KIRs and their cognate HLA ligands set a threshold for NK cell cytotoxic activity. Moreover, it has been shown to critically influence the course of viral infections, associating with resolution of hepatitis C infection^{[30]}. In HIV, HLA/KIR combinations have been associated with the pace of disease progression^{[31,32]} and protection against disease acquisition^{[33,34]}. The mechanisms conferring this protection may include both NK cell education, or “licensing”, through inhibitory ligand activation^{[35]} and the direct interaction of KIRs with HIV-1-derived peptide motifs presented on HLA molecules. Specific viral protein/KIR combinations associate with differences in NK cell viral inhibition in vitro^{[36]} and HLA/KIR combinations confer differences in HIV control^{[37]}. The HLA/KIR inter-actions directed by specific HIV-derived peptides are further linked to measures of NK cell function in vitro and patterns of viral escape in population studies^{[38,39]}. This suggests that NK cell activation threshold, determined in part by the genetic variant of inhibitory receptor ligands, and the virus-associated peptides available for presentation on host HLA structure to define the protective efficacy of NK cell cytotoxic activity.

The presently proposed methods are based at least in part on exploiting these mechanisms to harness HIV-specific cytotoxic functions of allogeneic NK and γδ T cells, identified as graft-versus-virus effect, based on the presence of stress signals originated from HIV-infected cells and the mismatch of host HLA/donor MR profile, without the risk of a significant graft-versus-host reaction.

An exemplary overall flow of treatment is (which can be repeated as needed):

Pre-Conditioning from day −7 to day −1

Cell infusion on day 0

Post-conditioning from day 1 to day 30

An exemplary treatment outline for an HIV patient is as follows:

### Pre-Conditioning

Day −7 to day −4
Fludarabine 25 mg/m²/d IV over 30 minutes
Day −3 and −2
Cyclophosphamide 300 g/m²/d IV over 30 minutes
Day −2 and −1
Interferon Alpha 3×10⁶ IU/m²/d SC

### Adoptive Cell Therapy

Day 0
Pre-medication: Promethazine 25 mg and Diphenhydramine 25 mg IV over 30 minutes
NK cells 20-25×10⁶/kg and γδ T cells 5-10×10⁶ /kg IV over 60 minutes
Interleukin-2 6×10⁶ IU/m² SC

### Post-Conditioning

Day 1-4
Interleukin-2 6×10⁶ IU/m²/d SC
Day1-30
Celecoxib 200 mg/d PO
Aspirin 120-300 mg/d PO (unless counter-indicated)

### EXAMPLE 3A

Eliciting Immune Response in HIV Patient using Activated NK Cells Activated Lymphocyte Regimen in HIV Patient

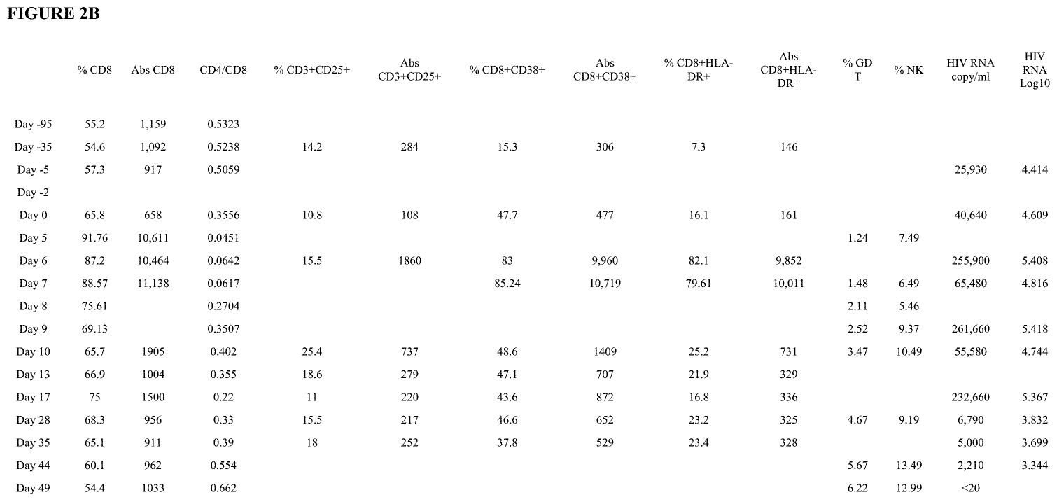

As shown in FIGS. 2A-B, a patient with uncontrolled viremia and no longer taking anti-retroviral therapy (ART) drugs due to tolerability and/or toxicity issues was treated using the activated NK cell regimen as follows:

RUM Pre-conditioning regimen:
Fludarabine 15 mg/m²/d from day −7 to day −3
Cyclophosphamide 500 mg/m²/d on day −2 and day −1
Interferon Alpha 3×10⁶ IU/m²/d on day −2 and day −1

Day 0 (activated NK infusion day):
20×10⁶ cells/kg activated NK cells
5×10⁶ cells/kg activated GDT cells
6×10⁶ IU/m² IL-2 SC injection

Day 1 to Day 5:
6×10⁶ IU/m² IL-2 SC injection

Day 1 to Day 40:
200 mg/d Celecoxib PO

The patient experienced self-limited fever, chills and joint pain that was alleviated by celecoxib between days 3 and 6. No other adverse reactions observed. At day 49 post activated NK treatment, the HIV viral load was determined to be <20 HIV RNA copy/ml, which is nearly undetectable (See FIG. 2A and FIG. 2B).

The patient's HLA profile did not match the HLA profile of the NK+GDT donor cells.

These examples provided herein demonstrate the surprising and unexpected ability of the activated cellular composition to treat patients with HIV and/or cancer.

While not wishing to be bound by theory, it was believed that since virally infected cells are essentially like cancer cells (i.e. foreign cells dividing or replicating uncontrollably), that treatment of the patient with the activated lymphocyte compositions and methods as described herein, would result in killing of the HIV infected cells, and then continue to elicit an immune response with the “vaccine effect” of the activated lymphocyte, and in this case activated NK plus activated gamma delta T cell treatment, and continue to control the viremia by the phagocytic capabilities of the activated NK cells. It is expected that the NK cells will destroy/eliminate the HIV virions as they circulate in the patient's peripheral blood.

After treatment as shown in FIG. 2, the HIV viral load is dramatically reduced, and the patient's CD4 and CD8 cell count is rebounding, along with the patient's total lymph count. This patient was also administered the post-infusion treatment of a COX-2 inhibitor administered on the infusion day, and every day for out to 40 days following infusion.

Additionally, it is expected that this treatment would be repeated periodically to boost the immune system response to any remaining virus/virions. Such periodic treatment can vary from once every month, to once every two months, to once every 3 months, to once every 4 months, to once every 5 months, to once every 6 months, or once every 7 months, or once every 8 months, or once every 9 months, or once every 10 months, or every 11 months, or once annually as a maintenance treatment for as long as the patient requires.

### EXAMPLE 3B

### Eliminating the HIV Reservoir

In view of the drastic reduction of HIV viral load described in FIG. 2 and Example 2A, it is believed that the present allogeneic activated lymphocytic treatments will also be effective for eliminating the latent HIV reservoir that is essentially untouchable by standard ART. After infection with HIV, there is a latent reservoir that could be stimulated to become “visible” to the activated lymphocytic cells (“allo-cells”) due to “incomplete ART”. There isn't one big event that activates the latent HIV reservoir—but instead the HIV in the reservoir will naturally replicate at some slow rate, they would not be able to fuse or infect new T cells if concurrently treated with an “incomplete ART” which would include an anti-HIV mAb or any other anti-retroviral that is a fusion or entry inhibitor that interferes with the infection process (these provide incomplete treatment and must be combined with another agent, in contrast to an agent that prevents viral expression and production). Examples of such small molecule HIV fusion or entry inhibitors include: bevirimat (DSB;PA-457); Vicriviroc, Maraviroc (a chemokine receptor antagonist” or a “CCR5 inhibitor”), T-20 (enfuvirtide, Fuzeon, developed by Roche and Trimeris), TRI-1144, and TRI-999 (See, Qian, K et al, Med Res Rev. 2009 March; 29(2):369-393, and Haggani and Tilton, Antiviral Res. 2013 May; 98(2):158-70). Similarly, examples of anti-HIV mAbs include those against CCR5 and a CD4, and specifically: Ibalizumab (trade name Trogarzo) is a non-immunosuppressive humanized monoclonal antibody that binds CD4; PRO 140 is a humanized monoclonal antibody targeted against the CCR5. During the brief period of time after viral burst and before fusing into a host CD4 T cell, the activated lymphocytic cells (“allo-cells”), as described in the present treatment methods (including the various pre-conditioning and post-conditioning options) would function to kill the HIV virus and prevent it from infecting new cells. Using this activated lymphocytic cell treatment and regimen, as described herein, in conjunction with “incomplete ART” regimen, will allow the latently infected cells to express the virus so that the virus can then be exposed to the killing by activated lymphocytic cells. The result will be actively killing all the virus-producing cells with activated lymphocytic cells, while protecting the new T cells from being infected because of the presence of the “incomplete ART” mAbs or small molecule inhibitors, as described above.

Additionally, in certain patients, it is expected that this treatment would be repeated periodically to boost the immune system response to any remaining virus/virions. Such periodic treatment can vary from once every month, to once every two months, to once every 3 months, to once every 4 months, to once every 5 months, to once every 6 months, or once every 7 months, or once every 8 months, or once every 9 months, or once every 10 months, or every 11 months, or once annually as a maintenance treatment for as long as the patient requires.

### Methods

A. Activation/Differentiation with Freshly Harvested NK Cells

Healthy donor derived resting peripheral blood NK cells were isolated from healthy donor leukapheresis material using Miltenyi NK Isolation Kit (negative selection)

Cells were seeded with 10⁶/ml cell concentration

12-well plate, 3×10⁶ cells/well

Basal Medium: Miltenyi GMP Grade NK Media

Experimental conditions:
1. Basal media
2. Basal media+IL-2 (500 IU/ml)
3. Basal media+FGFR1 (33 nM)
4. Basal media+IL-2 (500 IU/ml)+FGFR1 (33 nM)

Each condition was tested in triplicate.

Day 0 (seeding) cell count: 10⁶ total viable cell per ml

TABLE 6
24 hour cell count results (average of 3 in each group):

| | Total Cell Count/ml | Viability % | Live Cell Count/ml |
|---|---|---|---|
| 1 | 9.98 × 10⁵ | 97 | 9.68 × 10⁵ |
| 2 | 9.01 × 10⁵ | 98.5 | 8.88 × 10⁵ |
| 3 | 1.02 × 10⁶ | 98.9 | 10⁶ |
| 4 | 9.26 × 10⁵ | 97.2 | 9 × 10⁵ |

TABLE 7
48 hour cell count results (average of 3 in each group):

| | Total Cell Count/ml | Viability % | Live Cell Count/ml |
|---|---|---|---|
| 1 | 9.27 × 10⁵ | 95 | 8.8 × 10⁵ |
| 2 | 8.99 × 10⁵ | 97.2 | 8.73 × 10⁵ |
| 3 | 1.04 × l0⁶ | 98.4 | 1.02 × 10⁶ |
| 4 | 9.19 × 10⁵ | 95.9 | 8.81 × 10⁵ |

TABLE 8
72 hour cell count results (average of 3 in each group):

| | Total Cell Count/ml | Viability % | Live Cell Count/ml |
|---|---|---|---|
| 1 | 8.13 × 10⁵ | 91 | 7.39 × 10⁵ |
| 2 | 8.98 × 10⁵ | 97.0 | 8.71 × 10⁵ |
| 3 | 1.19 × 10⁵ | 98.1 | 1.16 × 10⁶ |
| 4 | 9.42 × 10⁵ | 94.9 | 8.93 × 10⁵ |

These data illustrate the stability and viability of freshly harvested NK cells as a source (that is commercially scalable) of cells for therapeutic compositions and applications as described in the methods herein.

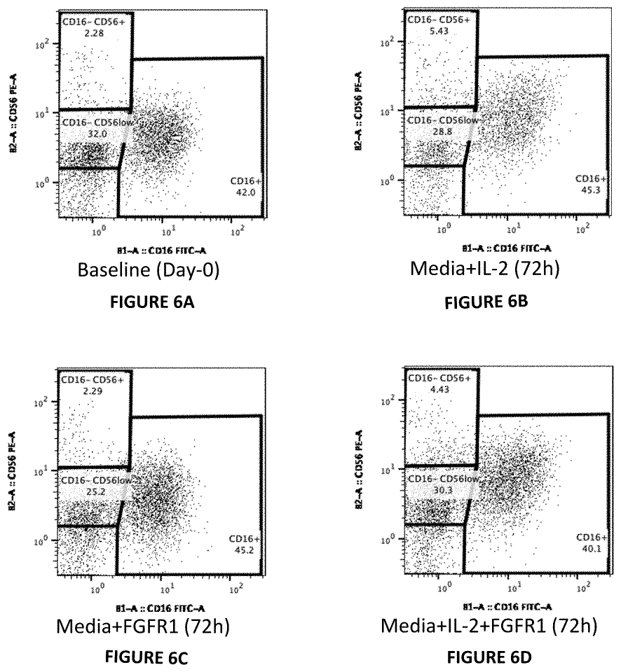

As shown in FIG. 6C, FGFR1 activation is superior to that of IL-2 alone, when evaluating viability and the total cell number over the course of 72 hours. As shown above in Tables 6-8, there is a slight expansion of cells in the FGFR1 group (see results for row 3). The addition of IL-2 to FGFR1 decreases the proliferative effect of FGFR1 alone. This is further reflected in FIGS. 6A-D, which compare cytotoxic cell numbers to regulatory/non-cytotoxic NK cell distribution at the end of 72 hours of activation. Three main populations have been gated in this analysis:

CD16−CD56+ NK population which is a regulatory subset;

CD16−CD56dim NK population which is a regulatory/resting subset;

CD16+NK population which is activated/cytotoxic subset.

Thus, comparing the “cytotoxic vs non-cytotoxic/regulatory population, the flow cytometry data illustrates that the FGFR1 activation subset (FIG. 6C) exhibits the biggest increase in cytotoxic NK cells (CD16+, or also CD16+CD56dim, or CD16−CD56dim), and the fewest regulatory NK cells. In the IL-2 only activation subset (FIG. 6B) there is a much greater number of regulatory NK cells, (CD16−CD56+), which could contribute to immune tolerance or immune exhaustion in an adoptive cell therapy setting. Thus, the FGFR1 or combination IL-2+FGFR1 activated NK populations are the most desirable for the present methods.

### B Activation/Differentiation of Cryopreserved NK Cells

Healthy donor derived resting peripheral blood NK cells were isolated from healthy donor leukapheresis material using Miltenyi NK Isolation Kit (negative selection, Miltenyi Biotec Inc.).

The cells were stored at −80C for 14 days before the experiment. They were stored in Cryostor CS10 cryopreservation solution, at a concentration of 5×10⁶/ml in 1.8 ml cryotubes.

Cells were thawed in 37° C. water bath and immediately seeded in the culture plate.

Cells were seeded with 10⁶/ml cell concentration, in a 12-well plate, 3×10⁶ cells/well.

Basal Medium: Miltenyi GMP Grade NK Media

Experimental conditions:
1. Basal media
2. Basal media+IL-2 (500 IU/ml)
3. Basal media+FGFR1 (33 nM)
4. Basal media+IL-2 (500 IU/ml)+FGFR1 (33 nM)

Each condition was tested in triplicate.

Day 0 (seeding) cell count: 10⁶ total viable cell per ml

TABLE 9
24 hour cell count results (average of 3 in each group):

| | Total Cell Count/ml | Viability % | Live Cell Count/ml |
|---|---|---|---|
| 1 | 8.71 × 10⁵ | 91 | 7.92 × 10⁵ |
| 2 | 8.16 × 10⁵ | 90.5 | 7.38 × 10⁵ |
| 3 | 8.11 × 10⁵ | 93 | 7.54 × 10⁵ |
| 4 | 8.44 × 10⁵ | 92.7 | 7.82 × 10⁵ |

TABLE 10
48 hour cell count results (average of 3 in each group):

| | Total Cell Count/ml | Viability % | Live Cell Count/ml |
|---|---|---|---|
| 1 | 8.10 × 10⁵ | 92 | 7.45 × 10⁵ |
| 2 | 7.99 × 10⁵ | 94.2 | 7.52 × 10⁵ |
| 3 | 8.01 × 10⁵ | 94.4 | 7.56 × 10⁵ |
| 4 | 7.91 × 10⁵ | 94 | 7.43 × 10⁵ |

TABLE 11
72 hour cell count results (average of 3 in each group):

| | Total Cell Count/ml | Viability % | Live Cell Count/ml |
|---|---|---|---|
| 1 | 6.73 × 10⁵ | 91 | 6.13 × 10⁵ |
| 2 | 7.87 × 10⁵ | 90.5 | 7.12 × 10⁵ |
| 3 | 8.97 × 10⁵ | 92.3 | 8.27 × 10⁵ |
| 4 | 9.51 × 10⁵ | 93.7 | 8.91 × 10⁵ |

These data illustrate the stability and viability of cryopreserved NK cells as another source (that is commercially scalable) of cells for therapeutic compositions and applications as described in the methods herein.

### Adverse Event Characterizations

The severity of AEs will be assessed using Common Terminology Criteria for Adverse Events (CTCAE) v5.0 Grades 1 through 5. If an event is not classified by CTCAE, the severity of the event will be graded by the investigator according to the scale below to estimate severity.

| | |
|---|---|
| Grade 1 | Mild; asymptomatic or mild symptoms; |
| | clinical or diagnostic observations only; |
| | intervention not indicated. |
| Grade 2 | Moderate; minimal, local or noninvasive |
| | intervention indicated; limiting age |
| | appropriate instrumental activities of daily living. |
| Grade 3 | Severe or medically significant but not |
| | immediately life-threatening; hospitalization |
| | or prolongation of hospitalization indicated; |
| | disabling; limiting self- care activities of daily living. |
| Grade 4 | Life-threatening consequences; urgent |
| | intervention indicated. |
| Grade 5 | Death related to AE. |

### Standard Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

All references cited herein are incorporated by reference to the same extent as if each individual publication, database entry (e.g. Genbank sequences or GenelD entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GenelD entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present embodiments are not to be limited in scope by the specific embodiments described herein. Indeed, various modifications of the embodiments in addition to those described herein will become apparent to those skilled in the art from the foregoing description and the accompanying figures. Such modifications are intended to fall within the scope of the embodiments and appended claims.

1. A method of treating a patient with HIV, cancer, a viral infection, or a bacterial infection, the method comprising administering an effective amount of a lymphocytic cellular composition comprising activated natural killer (NK) cells to the patient.

2-5. (canceled)

6. The method of claim 1, wherein the cellular composition further comprises activated gamma delta T cells (GDT cells).

7. The method of claim 1, wherein the cellular composition further comprises invariant natural killer T cells (iNKT cells).

8. The method of claim 1, wherein the cellular composition further comprises CD3⁺ T cells.

9. The method of claim 1, wherein prior to administration to the patient, the lymphocytic cellular composition is activated by mixing/incubating/contacting the cells with at least one cytokine and optionally soluble fibroblast growth factor receptor 1 (sFGFR1).

10. The method of claim 9, wherein the cytokine is selected from the group consisting of IL-2, IL-15, IL-21, Flt3-L, stem cell factor (SCF), IL-7, IL-12, and IL18, and any combination thereof.

11-12. (canceled)

13. The method of claim 1, wherein the method further comprises pre-conditioning the patient prior to administering the lymphocytic cellular composition by administering at least one lympho-suppressive agent, chemotherapeutic agent, or immunosuppressive agent for between 3-5 days prior to administering the lymphocytic cellular composition.

14. The method of claim 13, wherein the lympho-suppressive or chemotherapeutic agent comprises any one of 6TG, 6-MMP, one or more purine analogues selected from the group consisting of clofarabine, fludarabine, and cytarabine, or any combination thereof.

15. (canceled)

16. The method of claim 13, wherein the chemotherapeutic or immunosuppressive agent comprises any cyclophosphamide, rituximab, a steroid, or any combination thereof.

17-19. (canceled)

20. The method of claim 13, wherein the method further comprises administering to the patient interferon-alpha (IFN-α), or a biological equivalent thereof, at a dosage of from about 1×10⁶ IU/m²/d to about 10×10⁶ IU/m²/d, two days and/or 1 day prior to administering the lymphocytic cellular composition.

21. The method of claim 1, wherein the method further comprises administering to the patient IL-2 on the day of administration of the lymphocytic cellular composition and on the first day following administration of the lymphocytic cellular composition, and optionally continuing for between 3-14 additional days following administration of the lymphocytic cellular composition, in a dosage of about 3-6×10⁶ IU/m² per dose.

22. The method of claim 1, wherein the method further comprises administering to the patient a COX-2 inhibitor or a nonsteroidal anti-inflammatory drug (NSAID), or a combination thereof, on the day of administration of the lymphocytic cellular composition, and continuing for between at least 14 and 60 days following administration of the lymphocytic cellular composition.

23. (canceled)

24. The method of claim 1, wherein the activated NK cells are allogeneic to the patient.

25. The method of claim 1, wherein the NK cells are not HLA-matched with the patient.

26. (canceled)

27. The method of claim 1, wherein the viral infection is selected from the group consisting of viral infections caused by retroviruses (e.g., human T-cell lymphotrophic virus (HTLV) types I and II and human immunodeficiency virus (HIV)), herpes viruses (e.g., herpes simplex virus (HSV) types I and II, Epstein-Ban virus and cytomegalovirus), arenaviruses (e.g., lassa fever virus), paramyxoviruses (e.g., morbillivirus virus, human respiratory syncytial virus, and pneumovirus), adenoviruses, bunyaviruses (e.g., hantavirus), coronaviruses, filoviruses (e.g., Ebola virus), flaviviruses (e.g., hepatitis C virus (HCV), yellow fever virus, and Japanese encephalitis virus), hepadnaviruses (e.g., hepatitis B viruses (HBV)), orthomyoviruses (e.g., Sendai virus and influenza viruses A, B and C), papovaviruses (e.g., papillomaviruses), picornaviruses (e.g., rhinoviruses, enteroviruses and hepatitis A viruses), poxviruses, reoviruses (e.g., rotaviruses), togaviruses (e.g., rubella virus), and rhabdoviruses (e.g., rabies virus), and any combination thereof.

28-34. (canceled)

36. A method of activating NK cells, the method comprising contacting a cellular composition comprising the NK cells in vitro with at least one cytokine and optionally soluble fibroblast growth factor receptor 1 (sFGFR1).

37. The method of claim 36, wherein the cytokine is selected from the group consisting of IL-2, IL-15, IL-21, Flt3-L, stem cell factor (SCF), IL-7, IL-12, and IL18, and any combination thereof.

38-43. (canceled)

44. A cytokine and optionally soluble fibroblast growth factor receptor 1 (sFGFR1) activated lymphocytic cellular composition comprising activated natural killer (NK) cells.

45. The composition of claim 44, wherein the cytokine activated composition a IL-2, IL-15, IL-21, Flt3-L, stem cell factor (SCF), IL-7, IL-12, and IL18, and any combination thereof, activated composition.

46. The composition of claim 44, wherein the composition further comprises gamma delta T cells (GDT cells), invariant natural killer T cells (iNKT cells), and/or CD3⁺ T cells.

47-50. (canceled)


SEQUENCE LISTING (1 sequence)

SEQ ID NO: 1 - DNA, 7133 bp, Artificial Sequence
  synthetic sequence
taatacgactcactataggggaattgtgagcggataacaattcccctctagaaataattttgtttaactttaagaaggagatataccatgcatcaccatcaccatcactcccctatactaggttattggaaaattaagggccttgtgcaacccactcgacttcttttggaatatcttgaagaaaaatatgaggagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatgaaaacctgtattttcagggcatgaggccgtccccgaccttgcctgaacaagatgctctcccctcctcggaggatgatgatgatgatgatgactcctcttcagaggagaaagaaacagataacaccaaaccaaaccccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtggagcggtcccctcaccggcccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaagactgctggagttaataccaccgacaaagagatggaggtgcttcacttaagaaatgtctcctttgaggacgcaggggagtatacgtgcttggcgggtaactctatcggactctcccatcactctgcatggttgaccgttctggaagccctggaagagaggccggcagtgatgacctcgcccctgtacctggagtaaggatccgctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcacgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagggcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaat